SEQUENCE LISTING (44 sequences)

SEQ ID NO: 1 - DNA, 2704 bp, Homo sapiens
ggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcgtaattccatccactgctgagaaatctcctcaaacccagaaggtttaatcacttcatcccaaaaatgggattgtgaatgtcagcaaaccataaaaaaagtgcttagaagtattcctatagaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacgtgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctactatgcggcccggcatggtggctcacacctgtaatcccagcactttgggaggccaaggtgggtgggtcatctgaggtcaggagttcaagaccagcctggccaacatggtgaaaccccatctctactaaagatcaaaatttgctgagcgtggtggtgtgcacctgtatcccagctactcgagaggccaaggcatgagaatcgcttgaacctggaggtgaggttgcagtgagctgagatggcaccactgcactccggcctaggcaacgagagcaaaactccaatacaaacaaacaaacaaacacctgtgctaggtcagtctggcacgtaagatgaacatccctaccaacacagagctcaccatctcttatacttaagtgaaaaacatggggaaggggaaaggggaatggctgcttttgatatgttccctgacgcatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacataaaaaaaaaaaaaaaaaaaa

SEQ ID NO: 2 - DNA, 1516 bp, Mus musculus
ttcaagttccacgttccctactgctaagagtcttagcttacaaaagatattcttgtaagccaagtgtgaagttaatcacgacaaccaaaggtttgctaacatagaggaagagctctcatcaataggggaacagaaagtctcagcgacaagcttatgaaagaatggctgtctcaagggctccaacacccgactccgcctgtcagaggatggtctggctctttccacttgtcttctgcctcggctcagggagtgaagtttcacagagcagctcagacccccagctaatgaatggcgttctaggagagtctgcagttcttcctctaaagcttcctgcagggaagatagccaatatcatcatctggaattatgaatgggaagcgtcacaagtcactgccctcgttatcaacctaagtaatcctgaaagtccacaaatcatgaacactgatgtaaagaagagactgaacatcacccagtcctactccctgcaaatcagcaaccttaccatggcagacacaggatcatacactgcgcagataaccacaaaggactctgaagtgatcaccttcaaatatattctgagggtctttgaacgattgggtaacttagaaactaccaactatactctcctgctagagaatgggacctgccagatacacctggcctgtgttttgaagaatcaaagtcaaactgtctcagttgagtggcaagccacaggaaacatctctttaggaggaccaaatgtcactatcttttgggacccgaggaattctggtgaccagacttacgtctgcagagccaagaatgctgtcagcaatttgtcagtctctgtttcgacccagagtctctgcaaaggggttctaactaatccaccctggaatgcagtatggtttatgactacaatttcaataatcagtgcagtcatactcatctttgtgtgctggagcatacatgtttggaagagaagaggttctcttcctttgactagccaacatccagagtcctcccagagcacagatggcccaggctctccagggaacactgtgtatgcacaagtcactcgtccaatgcaggaaatgaaaatcccaaaacctatcaaaaatgactccatgacaatttactccatagttaatcattccagagaggaaacagtggctttaaccggctataaccaacccattaccctgaaggttaacactttaatcaactataactcctgaaggaagagcactgcagtgacttgaggaaattaaacaatgctgtcaccacagctctggcttagattaatgaagtcagcatctctggagattgagcgctgccatttgcattgttcaaacgctttctaggtggtatggtgagatgccagagggctaagggccattatagcagggtagtttgactaggaatacataagatagaaagcctagaatcgtatcattgaaagggacaatggacctaagagaagtggaataaaattgtgtcacacaaaaaaaaaaaaaaaaaaaaaaaaaaagcttgt

SEQ ID NO: 3 - DNA, 1408 bp, Homo sapiens
gaattcgaattcgggactttccagaaggaccacagctcctcccgtgcatccactcggcctgggaggttctggattttggctgtcgagggagtttgcctgcctctccagagaaagatggtcatgaggcccctgtggagtctgcttctctgggaagccctacttcccattacagttactggtgcccaagtgctgagcaaagtcgggggctcggtgctgctggtggcagcgcgtccccctggcttccaagtccgtgaggctatctggcgatctctctggccttcagaagagctcctggccacgtttttccgaggctccctggagactctgtaccattcccgcttcctgggccgagcccagctacacagcaacctcagcctggagctcgggccgctggagtctggagacagcggcaacttctccgtgttgatggtggacacaaggggccagccctggacccagaccctccagctcaaggtgtacgatgcagtgcccaggcccgtggtacaagtgttcattgctgtagaaagggatgctcagccctccaagacctgccaggttttcttgtcctgttgggcccccaacatcagcgaaataacctatagctggcgacgggagacaaccatggactttggtatggaaccacacagcctcttcacagacggacaggtgctgagcatttccctgggaccaggagacagagatgtggcctattcctgcattgtctccaaccctgtcagctgggacttggccacagtcacgccctgggatagctgtcatcatgaggcagcaccagggaaggcctcctacaaagatgtgctgctggtggtggtgcctgtctcgctgctcctgatgctggttactctcttctctgcctggcactggtgcccctgctcagggaaaaagaaaaaggatgtccatgctgacagagtgggtccagagacagagaacccccttgtgcaggatctgccataaaggacaatatgaactgatgcctggactatcagtaaccccactgcacaggcacacgatgctctgggacataactggtgcctggaaatcaccatggtcctcatatctcccatgggaatcctgtcctgcctcgaaggagcagcctgggcagccatcacaccacgaggacaggaagcaccagcacgtttcacacctcccccttccctctcccatcttctcatatcctggctcttctctgggcaagatgagccaagcagaacattccatccaggacactggaagttctccaggatccagatccatggggacattaatagtccaaggcattccctcccccaccactattcataaagtattaaccaactggcaccaaaaaaaaaaaaaaaaaaaaaaaaaaaaaaaaaaaaaaaaaaaaaaaaaaaaaaagggcggccgcccg

SEQ ID NO: 4 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 5 - protein, 351 aa, Mus musculus
MAVSRAPTPDSACQRMVWLFPLVFCLGSGSEVSQSSSDPQLMNGVLGESAVLPLKLPAGKIANIIIWNYEWEASQVTALVINLSNPESPQIMNTDVKKRLNITQSYSLQISNLTMADTGSYTAQITTKDSEVITFKYILRVFERLGNLETTNYTLLLENGTCQIHLACVLKNQSQTVSVEWQATGNISLGGPNVTIFWDPRNSGDQTYVCRAKNAVSNLSVSVSTQSLCKGVLTNPPWNAVWFMTTISIISAVILIFVCWSIHVWKRRGSLPLTSQHPESSQSTDGPGSPGNTVYAQVTRPMQEMKIPKPIKNDSMTIYSIVNHSREETVALTGYNQPITLKVNTLINYNS

SEQ ID NO: 6 - protein, 285 aa, Homo sapiens
MVMRPLWSLLLWEALLPITVTGAQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDVLLVVVPVSLLLMLVTLFSAWHWCPCSGKKKKDVHADRVGPETENPLVQDLP

SEQ ID NO: 7 - DNA, 19 bp, Artificial Sequence
  Description of Artificial Sequence JNF1 PRIMER
cagagtacgacacaatccc

SEQ ID NO: 8 - DNA, 20 bp, Artificial Sequence
  Description of Artificial Sequence JNF2 PRIMER
actccactgtggaaataccg

SEQ ID NO: 9 - DNA, 52 bp, Artificial Sequence
  Description of Artificial Sequence JNF3 PRIMER
ccagtgagcagagtgacgaggactcgagctcaagcttttttttttttttttt

SEQ ID NO: 10 - DNA, 18 bp, Artificial Sequence
  Description of Artificial Sequence JNF4 PRIMER
gaggactcgagctcaagc

SEQ ID NO: 11 - DNA, 18 bp, Artificial Sequence
  Description of Artificial Sequence JNF5 PRIMER
ccagtgagcagagtgacg

SEQ ID NO: 12 - DNA, 20 bp, Artificial Sequence
  Description of Artificial Sequence JNF6 PRIMER
atcctttggcagctcacagg

SEQ ID NO: 13 - DNA, 19 bp, Artificial Sequence
  Description of Artificial Sequence JNF7 PRIMER
cttcacagagcttcctggc

SEQ ID NO: 14 - DNA, 20 bp, Artificial Sequence
  Description of Artificial Sequence JNF12 PRIMER
gagtcttgctaccaggaggg

SEQ ID NO: 15 - DNA, 20 bp, Artificial Sequence
  Description of Artificial Sequence JNF13 PRIMER
gacacagaagtgtgcatggc

SEQ ID NO: 16 - DNA, 20 bp, Artificial Sequence
  Description of Artificial Sequence JNF14 PRIMER
tggctcacacctgtaatccc

SEQ ID NO: 17 - DNA, 20 bp, Artificial Sequence
  Description of Artificial Sequence JNF15 PRIMER
catctcagctcactgcaacc

SEQ ID NO: 18 - DNA, 20 bp, Artificial Sequence
  Description of Artificial Sequence JNF16 PRIMER
gactcggtctctttattggg

SEQ ID NO: 19 - DNA, 20 bp, Artificial Sequence
  Description of Artificial Sequence JNF17 PRIMER
aagctcagcatagtgctggg

SEQ ID NO: 20 - DNA, 20 bp, Artificial Sequence
  Description of Artificial Sequence JNF18 PRIMER
gatttctcagcagtggatgg

SEQ ID NO: 21 - DNA, 32 bp, Artificial Sequence
  Description of Artificial Sequence LLEWELLYN4 PRIMER
cccaagcttccagagagcaatatggctggtcc

SEQ ID NO: 22 - DNA, 20 bp, Artificial Sequence
  Description of Artificial Sequence JNF19 PRIMER
acctgccagatacacctggc

SEQ ID NO: 23 - DNA, 20 bp, Artificial Sequence
  Description of Artificial Sequence JNF20 PRIMER
agacttacgtctgcagagcc

SEQ ID NO: 24 - DNA, 20 bp, Artificial Sequence
  Description of Artificial Sequence JNF21 PRIMER
gctggagcatacatgtttgg

SEQ ID NO: 25 - DNA, 20 bp, Artificial Sequence
  Description of Artificial Sequence JNF22 PRIMER
ttaaccttcagggtaatggg

SEQ ID NO: 26 - DNA, 20 bp, Artificial Sequence
  Description of Artificial Sequence JNF23 PRIMER
gaacaatgcaaatggcagcg

SEQ ID NO: 27 - DNA, 20 bp, Artificial Sequence
  Description of Artificial Sequence JNF33 PRIMER
acaggacaagaaaacctggc

SEQ ID NO: 28 - DNA, 21 bp, Artificial Sequence
  Description of Artificial Sequence JNF34 PRIMER
cgaaataacctatagctggcg

SEQ ID NO: 29 - DNA, 20 bp, Artificial Sequence
  Description of Artificial Sequence JNF35 PRIMER
tcctgcattgtctccaaccc

SEQ ID NO: 30 - DNA, 19 bp, Artificial Sequence
  Description of Artificial Sequence JNF36 PRIMER
ggcagatcctgcacaaggg

SEQ ID NO: 31 - DNA, 20 bp, Artificial Sequence
  Description of Artificial Sequence JNF37 PRIMER
atcttgcccagagaagagcc

SEQ ID NO: 32 - DNA, 20 bp, Artificial Sequence
  Description of Artificial Sequence JNF38 PRIMER
gaagctgatgctggcatggg

SEQ ID NO: 33 - DNA, 20 bp, Artificial Sequence
  Description of Artificial Sequence JNF39 PRIMER
catatctcccatgggaatcc

SEQ ID NO: 34 - DNA, 21 bp, Artificial Sequence
  Description of Artificial Sequence JNF40 PRIMER
ctggcctaaggactttcaggt

SEQ ID NO: 35 - DNA, 33 bp, Artificial Sequence
  Description of Artificial Sequence  forward primer HIIIDS4fp
cccaagcttccagagagcaatatggctggttcc

SEQ ID NO: 36 - DNA, 35 bp, Artificial Sequence
  Description of Artificial Sequence reverse primer DS4Bamrp
cgcggatccgaggaatctgggtcatcagcagcacc

SEQ ID NO: 37 - DNA, 30 bp, Artificial Sequence
  Description of Artificial Sequence forward primer Apex2Kpn1FP
ccgggtaccaacagaaagtctcagcgacaa

SEQ ID NO: 38 - DNA, 29 bp, Artificial Sequence
  Description of Artificial Sequence reverse primer Apex2BamRP
cgcggatcccagggtggattagttagaac

SEQ ID NO: 39 - DNA, 63 bp, Artificial Sequence
  Description of Artificial Sequence forward primer SpeFlagA1fp
cggactagtgactacaaggacgacgatgacaagtctggacccgtgaaagagctggtcggttcc

SEQ ID NO: 40 - DNA, 39 bp, Artificial Sequence
  Description of Artificial Sequence reverse primer A1Xbarp
tgctctagacactgctgtctagataacattctcataggc

SEQ ID NO: 41 - DNA, 63 bp, Artificial Sequence
  Description of Artificial Sequence Apex-2 forward primer SpeFlagA2fp
cggactagtgactacaaggacgacgatgacaagagtgaagtttcacagagcagctcagacccc

SEQ ID NO: 42 - DNA, 39 bp, Artificial Sequence
  Description of Artificial Sequence reverse primer A2xbarp
tgctctagacaagtcactgcagtgctcttccttcaggag

SEQ ID NO: 43 - protein, 8 aa, Artificial Sequence
  Description of Artificial Sequence FLAG tag peptide
DYKDDDDK

SEQ ID NO: 44 - protein, 24 aa, Artificial Sequence
  Description of Artificial Sequence FLAG tag peptide
MPMGSLQPLATLYLLGMLVASCLG

(19) United States
(12) Patent Application Publication
(10) Pub. No.: US 2002/0123617 A1
(43) Pub. Date: Sep. 5, 2002

(54) Novel immunoglobulin superfamily members of APEX-1, APEX-2 and APEX-3 and uses thereof

(76) Inventors: Gary C. Starling, Lawrenceville, NJ (US); Joshua N. Finger, San Marcos, CA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/745,605

(22) Filed: Dec. 22, 2000

Related U.S. Application Data
(60) Provisional application No. 60/172,025, filed on Dec. 23, 1999.

(51) Int. Cl. C07K 16/18; C07H 21/04; C12P 21/02; C12N 5/06

(52) U.S. Cl. 530/388.1; 536/23.53; 435/326; 435/320.1; 435/69.1

(57) ABSTRACT

The present invention provides the nucleotide and amino acid sequences of apex-1, -2, and -3, encoding novel APEX proteins APEX-1, APEX-2, and APEX-3, which are novel gene members of the CD2 subgroup of the immunoglobulin superfamily.

This application is based on a provisional application, U.S. Ser. No. 60/172,025, filed Dec. 23, 1999, the contents of which are hereby incorporated by reference in their entirety into this application.

Throughout this application, various publications are referenced. The disclosures of these publications in their entireties, are hereby incorporated by reference into this application, in order to more fully describe the state of the art, as known to those skilled therein, as of the date of invention, described and claimed herein.

1. Field of the Invention

The present invention relates to novel gene members encoding proteins of the immunoglobulin superfamily designated Antigen Presenting cell EXpression (APEX), including APEX-1, APEX-2, and APEX-3.

2. Background of the Invention

The CD2 subgroup of the immunoglobulin (Ig) superfamily consists primarily of cell-surface receptors that regulate adhesion among different leukocytes and generate co-stimulatory signals. This subgroup consists of CD2 (LFA-2), CD58 (LFA-3), CD48, CD59, CD84, Ly9, 2B4, and CDw150 (SLAM). All Ig family members exhibit structural similarities including two or more extracellular Ig-like domains, a transmembrane domain or a glycosylphosphatidylinositol (GPI)-anchor moiety. Family members which span the membrane also have a cytoplasmic domain which may, or may not, have specific SH2 domain binding motifs. Members of this family mediate diverse biological events including leukocyte proliferation, differentiation, migration, and activation (Williams, A. F. and Barclay, A. N., 1988 Ann. Rev. Immunol. 6:381-405).

CD2 was one of the first cell-adhesion molecules to be implicated in T-cell activation. In the early phase of the immune response, CD2 seems to facilitate antigen-independent activation and may be important in allowing the T-cell receptor (TCR) to sample different antigen-MHC complexes (Hahn, W. C., et al 1993 in: Lymphocyte Adhesion Molecules pp. 105-132, ed. Y. Shimizu, R. G. Landes Company). In addition to activation of both naive and memory T helper cells (Wingren, A. G., et al 1995 Critical Reviews in Immunology 15:235-253), interaction of CD2 with its ligand (mostly CD58 in humans, CD48 in rodents) results in the secretion of inflammatory cytokines (e.g. IL-1 and TNF). These inflammatory cytokines then recruit other immune cells to the site of injury or infection by upregulating adhesion molecule expression (Hahn, W. C., et al, 1993 in: Lymphocyte Adhesion Molecules pp. 105-132, ed. Y. Shmizu, R. G. Landes Company).

More recently, CD84, Ly9, 2B4 and SLAM have been shown to be structurally similar to the extracellular domains of CD2 family members, but contain slight differences (Angel de la Fuente, M., et al, 1997; Sandrin, M. S., et al, 1996; Cocks, B. G., et al, 1995). CD84 and SLAM each contain a V-set domain which lacks the usually conserved cysteine residues while the second domain is a truncated C2-set (tC2) domain containing conserved cysteine residues. Ly9 contains four Ig-like domains of the order V-tC2-V-tC2. All three molecules contain a cytoplasmic domain consisting of several SH2 domain binding motifs of the primary structure Y-X-X-hydrophobic (Songyang, Z., et al 1993 Cell 72:767-778). When these tyrosine residues are phosphorylated, they may become potential docking sites for kinases or other proteins. These kinases can act to phosphorylate other proteins and subsequently activate gene transcription. SLAM and 2B4 contain a motif T-X-Y-X-X-I/V, which is thought to be responsible for binding to SHP-2 kinase (Tangye, S. G., et al 1999 J. Immunol. 162:6981-6985). 2B4 is a receptor which positively regulates the activity of natural killer cells. Recent work has shown that 2B4 is a ligand for CD48 (Brown et. al. 1998 J. Exp. Med. 188:2083-2090), further demonstrating that members of this family of molecules are able to bind to other members of this family.

CD84, Ly9, and SLAM expression is predominantly restricted to hematopoietic tissues with highest levels in the spleen, lymph node and peripheral blood leukocytes (PBL). SLAM is expressed on activated T cells and immature thymocytes (Cocks, B. G., et al 1995 Nature 376:260-263), and is also found on activated antigen presenting cells (APCs).

CD84 and Ly9 functions have not been elucidated to date. SLAM, on the other hand, has been shown to enhance antigen-specific proliferation and cytokine production by CD4+T cells. Specifically, antibodies against SLAM strongly upregulate IFN-γ in both Th1 and Th2 clones, but do not induce IL-4 and IL-5 in Th1 clones. In addition, SLAM potentiates T-cell expansion in a CD28-independent manner (Cocks, B. G., et al 1995 Nature 376:260-263).

The present invention relates to the discovery of three new APEX protein members of the Ig superfamily, APEX-1, -2 and -3. The predicted products of the new apex-1, apex-2, and apex-3 Ig genes show homology to the CD2 subgroup, and may be classified as a marker of that subgroup, based on sequence homology.

### SUMMARY OF THE INVENTION

The acronym “APEX” or “apex” stands for Antigen Presenting cell EXpression, although the transcript expression pattern of the apex genes is not restricted to APCs. For convenience, the italicized term apex refers herein to an apex nucleic acid molecule or nucleotide sequences (gene). The capitalized term APEX refers herein to an APEX polypeptide (protein).

The present invention provides three novel nucleic acid molecules, designated apex-1, apex-2, and apex-3, and fragments and derivatives thereof (e.g. mutants, variants, homologues), having APEX activity, each having a nucleotide sequence which encodes a new APEX protein member of the immunoglobulin superfamily. In a particular aspect, apex-1 is described by SEQ ID NO: 1; apex-2 is described by SEQ ID NO: 2; and apex-3 is described by SEQ ID NO: 3.

In addition, the invention features nucleotide sequences that hybridize under stringent conditions to SEQ ID NO: 1, 2, or 3. The nucleic acid molecules of the invention further include portions of the apex sequences, such as fragments, oligonucleotides, or portions thereof, and peptide nucleic acids (PNA), and antisense molecules thereof which may be used to detect levels of apex transcripts that occur in a cell.

The invention provides isolated nucleic acid molecules and recombinant nucleic acid molecules having the apex sequences of the invention, and methods for uses thereof. The invention further provides isolated polypeptides and recombinant polypeptides having the APEX sequences of the invention, and methods for uses thereof.

The invention also provides isolated and substantially purified polypeptides APEX-1, APEX-2, and APEX-3. The invention further provides diagnostic assays and kits for the detection of naturally occurring APEX-1, -2 or -3 or the nucleic acids encoding them. It provides for the use of substantially purified APEX-1, -2 or -3 to produce antibodies reactive against an APEX protein, which can be used to quantitate the amount of APEX proteins in biological samples, e.g., in biological fluids or biopsied tissues from a subject.

These APEX proteins can also be used to produce antagonists which will bind to APEX molecules on the surface of tumor cells in vivo or in vitro, and as molecular weight markers. Substantially purified APEX proteins, or their fragments having APEX activity, may be useful as pharmaceutical compositions. For example, they may be used to inhibit cell adhesion.

The invention also relates to pharmaceutical compositions comprising antisense molecules capable of disrupting expression of apex genomic sequences, and agonists, antibodies, antagonists or inhibitors of the APEX proteins. These compositions are useful for the prevention or treatment of conditions associated with the presence or the expression of APEX proteins.

### BRIEF DESCRIPTION OF THE FIGURES

FIG. 1: A schematic representation of the general structure of the extracellular domains of the CD2 subgroup.

FIG. 2: The nucleotide sequence of apex-1 (SEQ ID NO: 1).

FIG. 3: The nucleotide sequence of apex-2. A portion of the nucleotide sequence of apex-2, beginning at thymine (t) at nucleotide 50 and ending at thymine (t) at nucleotide 1565 of FIG. 2 is shown in SEQ ID NO: 2.

FIG. 4: The nucleotide sequence of apex-3 (SEQ ID NO: 3).

FIG. 5: The amino acid sequence of APEX-1 protein (SEQ ID NO: 4).

FIG. 6: The amino acid sequence of APEX-2 protein (SEQ ID NO: 5).

FIG. 7: The amino acid sequence of APEX-3 protein (SEQ ID NO: 6).

FIG. 8: A and B: A Northern blot showing the detection of apex-1 transcripts in immune and non-immune tissues, as described in Example 1.

FIG. 9: A RT-PCR analysis showing detection of apex-1 transcripts in various cell types, as described in Example 1.

FIG. 10: The amino acid sequence of the extracellular domain of APEX-1 in the fusion protein APEX-1Ig. The predicted signal sequence (Met₁ to Ala₂₂) is shown in bold. The sequence from Ser₂₃ to Ser₂₂₅ corresponds to the extracellular domain of human APEX-1. The junction His₂₂₆ to Pro₂₂₇ sequence results from the BamHI restriction site and is followed by a sequence corresponding to the H—CH₂—CH₃ sequence from human IgG1.

FIG. 11: The amino acid sequence of the extracellular domain of APEX-2 in the fusion protein APEX-2Ig. The predicted signal sequence (Met₁ to Gly₂₉) is shown in bold and is expected to be cleaved in the mature protein. The sequence from Ser₃₀ to Trp₂₃₈ corresponds to the extracellular domain of murine APEX-2. The junction His₂₃₉ to Pro₂₄₀ sequence results from the BamHI restriction site and is followed by a sequence corresponding to the H—CH₂—CH₃ sequence of murine IgG2a.

FIG. 12: The SDS-PAGE of APEX-1Ig and APEX-2mIg fusion proteins, as described in Example 4. Proteins were run on a 12% Tris-Glycine gel under reducing conditions, and proteins visualized using coomassie blue staining.

FIG. 13: The SDS-PAGE of APEX-1Ig fusion protein expressed in COS and Sf9 cells, as described in Example 4.

FIG. 14: Western Blot analysis of the APEX-1 extracellular domains using a panel of anti-APEX-1 mAb, as described in Example 4. The mAbs were immunoblotted against the recombinant extracellular domain of APEX-1 under reducing and non-reducing conditions as shown.

FIG. 15: The amino acid sequence of fusion protein FLAG-APEX-1. The Met₁ to Gly₂₄ sequence is from human CD5 signal peptide and is expected to be cleaved in mature protein. The Asp₂₅ to Lys₃₂ sequence corresponds to the FLAG peptide sequence.

FIG. 16: The amino acid sequence of fusion protein FLAG-APEX-2. The Met₁ to Gly₂₄ sequence is from human CD5 signal peptide and is expected to be cleaved in mature protein. The Asp₂₅ to Lys₃₂ sequence corresponds to the FLAG peptide sequence.

### DETAILED DESCRIPTION OF THE INVENTION

Molecules of the Invention

In its various aspects, as described in detail below, the present invention provides APEX proteins, apex nucleic acid molecules (including apex nucleotide sequences, genomic and cDNA), recombinant DNA molecules, transformed host cells, generation methods, assays, antibodies, immunotherapeutic methods, transgenic animals, immunological and nucleic acid-based assays, and compositions.

For the sake of convenience, nucleic acid molecules having apex nucleotide sequences will be collectively referred to as the apex sequences, the nucleotide sequences of the invention, or apex. Additionally, APEX proteins will be collectively referred to as the APEX proteins, the proteins of the invention, or APEX.

Nucleic Acid Molecules

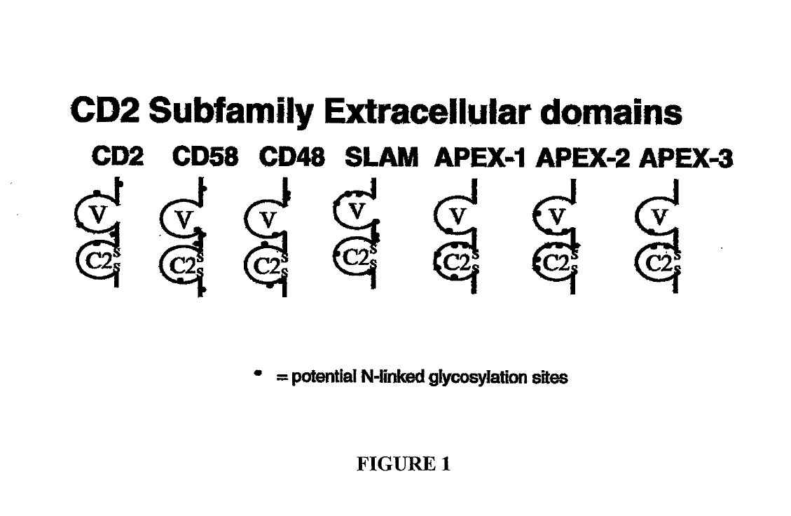

The present invention discloses the discovery of novel apex nucleotide sequences which encode full-length APEX polypeptides or fragments thereof which possess structural features shared with the CD2 subgroup (e.g., see FIG. 1) and are predicted to encode new members of the immunoglobulin superfamily. The structural features shared by the CD2 subgroup include a N-terminal signal peptide, an extracellular domain or region having Ig-like features, a hydrophobic transmembrane domain, and a C-terminal intracellular or cytoplasmic domain (e.g., see FIG. 1). Thus, similar to CD2 members, the APEX proteins may play a role as cell-surface receptors that regulate adhesion among different leukocytes and generate co-stimulatory signals.

One embodiment of the invention provides nucleic acid molecules that are DNA or RNA. Another embodiment provides nucleic acid molecules that exhibit significant sequence identity with the apex nucleotide sequences of the invention, such as molecules that have between about 60% to 99% sequence identity with the apex sequences of the invention. A preferred embodiment provides nucleic acid molecules that exhibit about 60% sequence identity, a more preferred embodiment provides molecules that have about 85% sequence identity, and the most preferred embodiment provides molecules that have about 95% to 99% sequence identity with the apex sequences of the invention. Accordingly, the present invention encompasses apex from any species, e.g., human, mouse and other mammals. The present invention also encompasses different alleles of the apex gene isolated from different subjects of the same mammalian species.

“Nucleotide sequences” as used herein refers to an oligonucleotides, or nucleotides, and fragments or portions thereof, and to DNA or RNA of genomic or synthetic origin which may be single- or double-stranded, and represents the sense or antisense strand.

“Peptide nucleic acid” (PNA) as used herein refers to a molecule which comprises an oligomer to which an amino acid residue, such as lysine, and an amino group have been added. These small molecules, also designated anti-sense and anti-gene agents, stop transcript elongation by binding to their complementary (template) strand of nucleic acid (Nielsen, P. E., et al., 1993) Anticancer Drug Des. 8:53-63).

As used herein, a nucleic acid molecule is said to be “isolated” when the nucleic acid molecule is substantially separated from contaminant nucleic acid molecules having sequences other than apex sequences. Additionally, isolated nucleic acid molecule refers to any RNA or DNA sequence, however constructed or synthesized. A skilled artisan can readily employ nucleic acid isolation procedures to obtain an isolated apex-encoding nucleic acid molecule, see Sambrook et al., Molecular Cloning (1989).

The nucleic acid molecules of the invention are preferably in isolated form, including DNA, RNA, DNA/RNA hybrids, and related molecules, nucleic acid molecules complementary to the APEX coding sequences or a part thereof, and those which hybridize to the nucleic acid sequences that encode the APEX proteins. The preferred nucleic acid molecules have a nucleotide sequence substantially identical to or complementary to the cDNA sequences herein disclosed. Specifically contemplated are genomic DNA, ribozymes, and antisense molecules, as well as nucleic acids based on an alternative backbone or including alternative bases, whether derived from natural sources or synthesized.

The invention further provides fragments of the APEX-encoding nucleic acid molecules of the present invention. As used herein, a fragment of an APEX-encoding nucleic acid molecule refers to a portion of the entire APEX-encoding sequence. The size of the fragment will be determined by its intended use. For example, if the fragment is chosen to encode an APEX-encoding extracellular domain, then the skilled artisan shall select the nucleotide fragment that is large enough to encode this functional domain(s) of the APEX protein. If the fragment is to be used as a nucleic acid probe or PCR primer, then the fragment length is chosen so as to minimize the number of false positives during probing or priming.

The present invention provides specific nucleic acid molecules encoding proteins designated APEX-1, -2, and -3 or fragments thereof having APEX activity. One embodiment encompassed by the present invention includes isolated nucleotide sequences of apex-1, -2, -3, as described in, e.g., SEQ ID NO.: 1, 2, or 3, respectively, or portions thereof. In particular, the nucleic acid molecules of the invention may be isolated full-length or partial cDNA molecules or oligomers of the apex -1, -2, or -3 sequences. The nucleic acid molecules of the invention each include the nucleotide sequences encoding all or portions of the signal peptide region, the extracellular domain, the transmembrane domain, and/or the intracellular domain of APEX -1, -2 or -3.

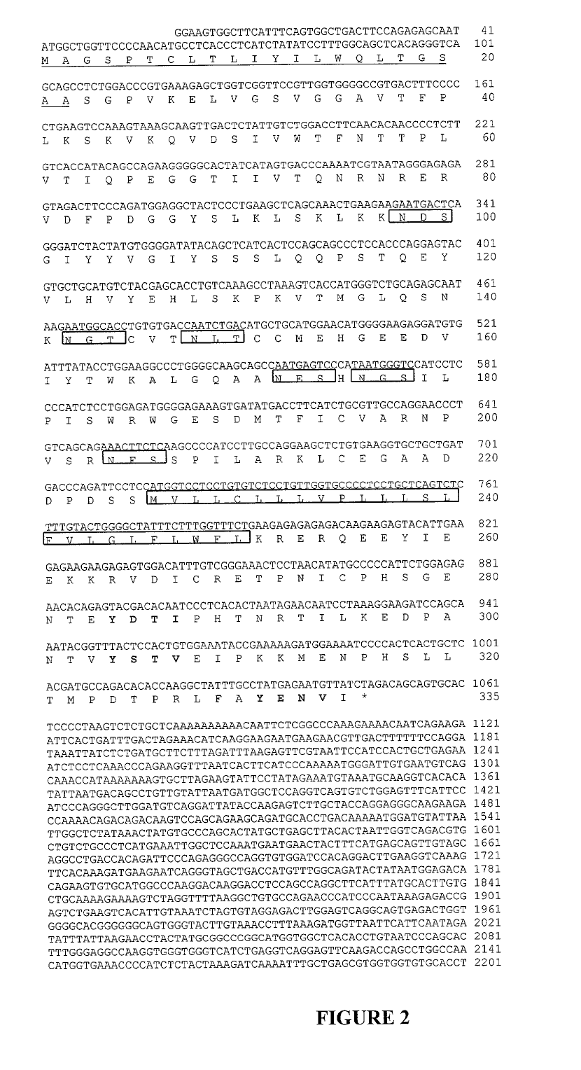

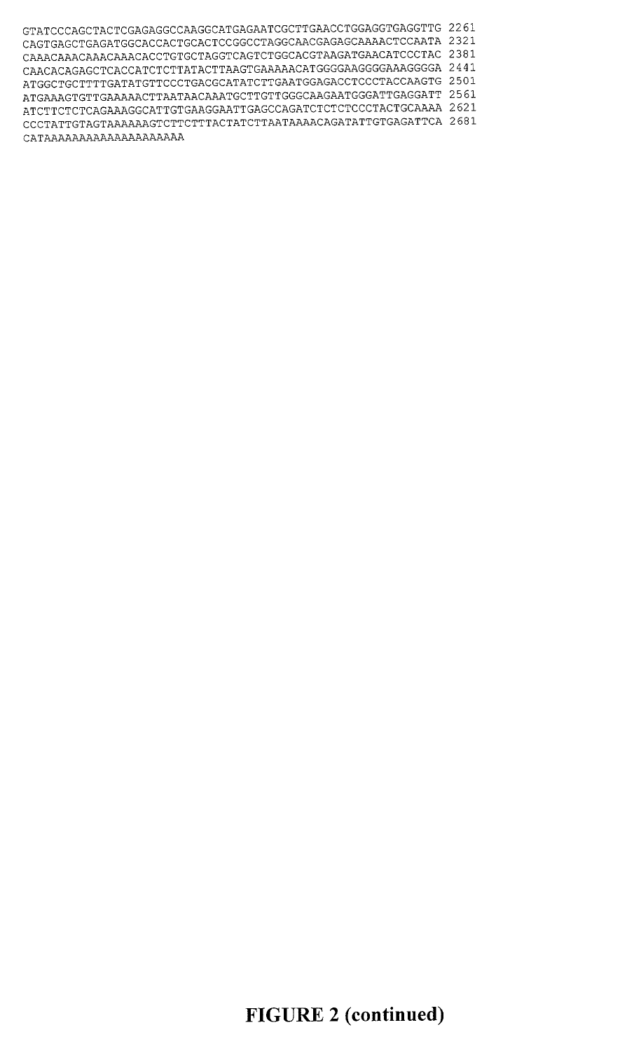

In one embodiment, apex-1 sequence as shown in SEQ ID NO. 1, comprises the coding sequence for APEX-1 protein between nucleotides 42 and 1049. The 1008 base pair coding sequence within apex-1 beginning at adenine (a) at position 42 and ending at guanine (g) at position 1049 can encode a 335 amino acid APEX-1 protein. The APEX-1 protein is comprised of a signal peptide, an extracellular domain, a transmenbrane domain, and a cytoplasmic domain (FIG. 2).

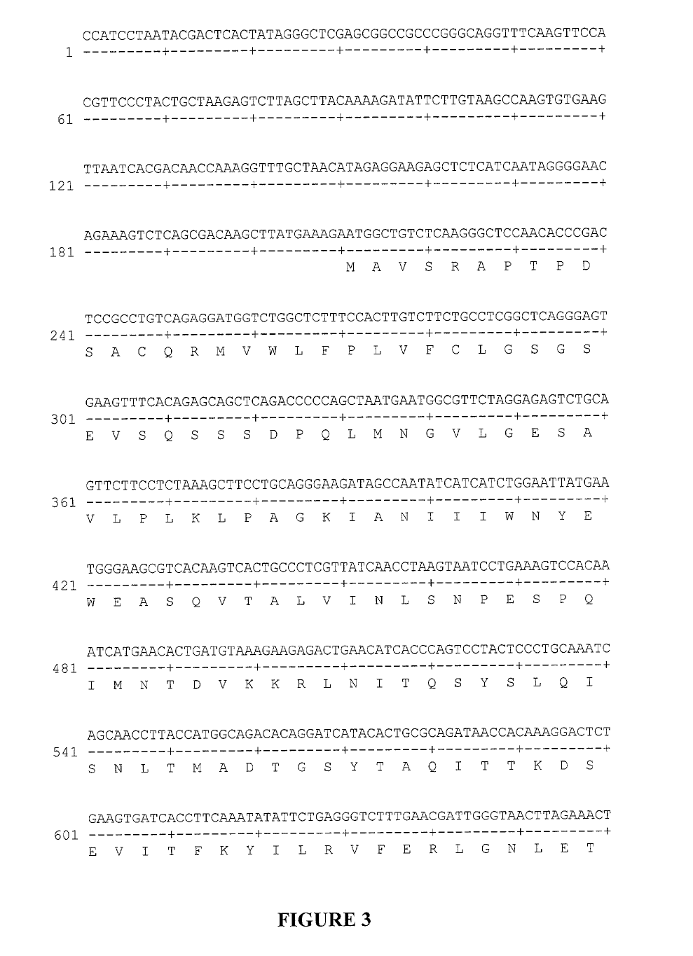

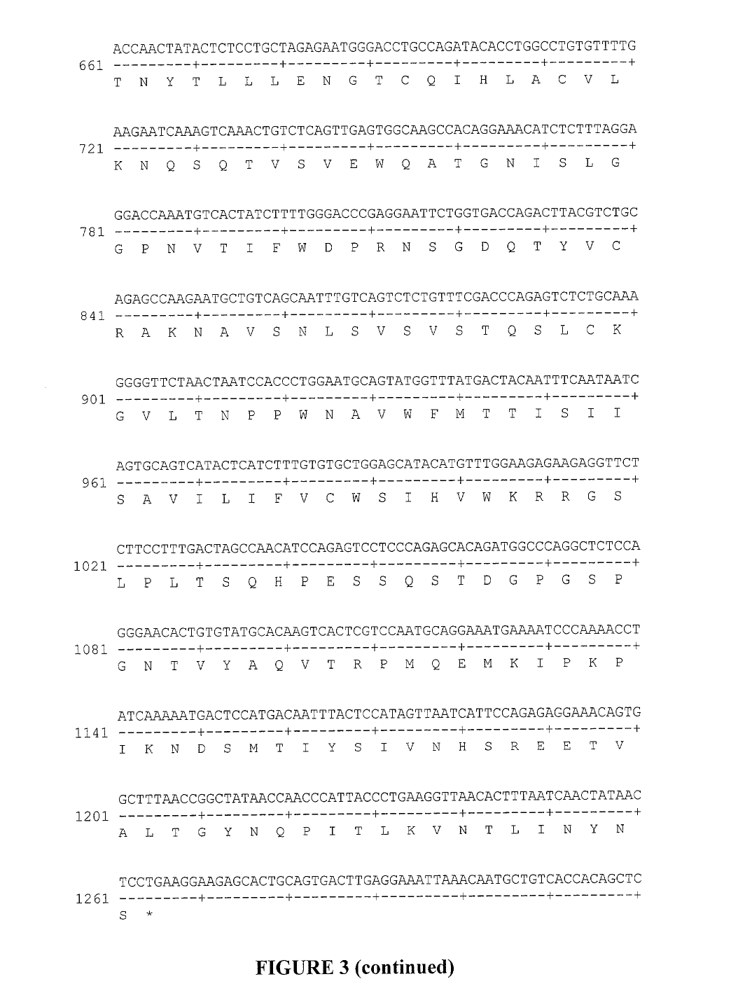

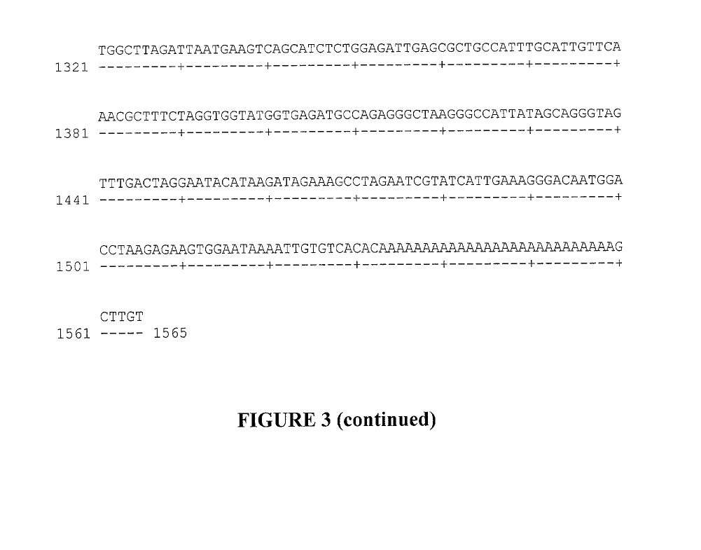

In another embodiment, apex-2 sequence as shown in SEQ ID NO. 2, comprises the coding sequence of APEX-2 protein between nucleotides 162 and 1217. The 1056 base pair coding region of apex-2 beginning at adenine (a) at position 162 and ending at adenine (a) at position 1217 as shown in SEQ ID NO.2 can encode a 351 amino acid APEX-2 protein The APEX-2 protein is comprised of a signal peptide, an extracellular domain, a transmembrane domain, and a cytoplasmic domain (FIG. 3).

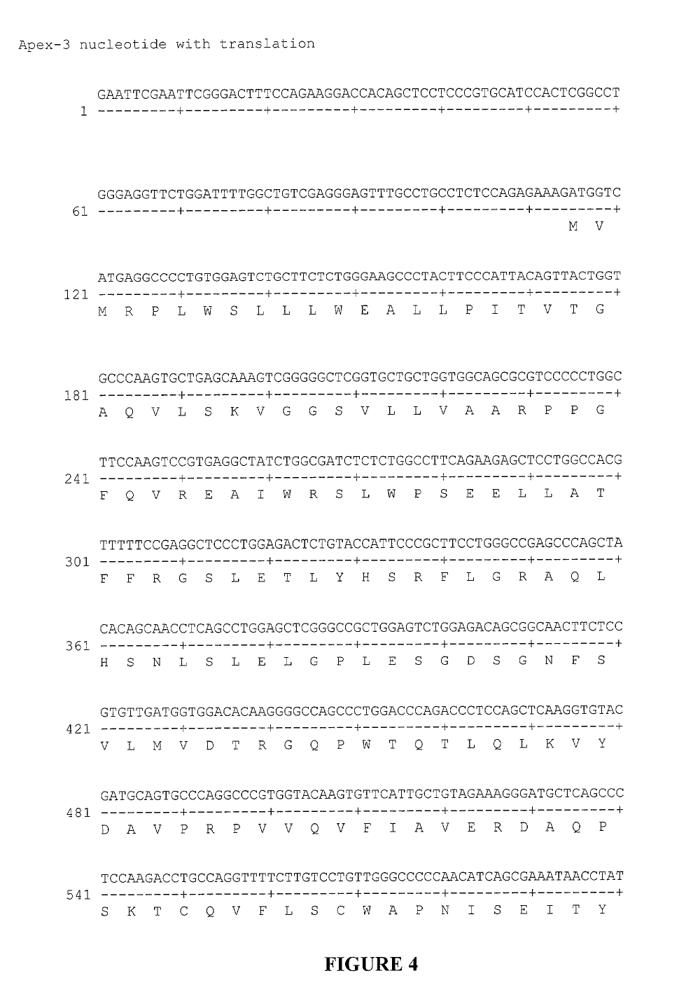

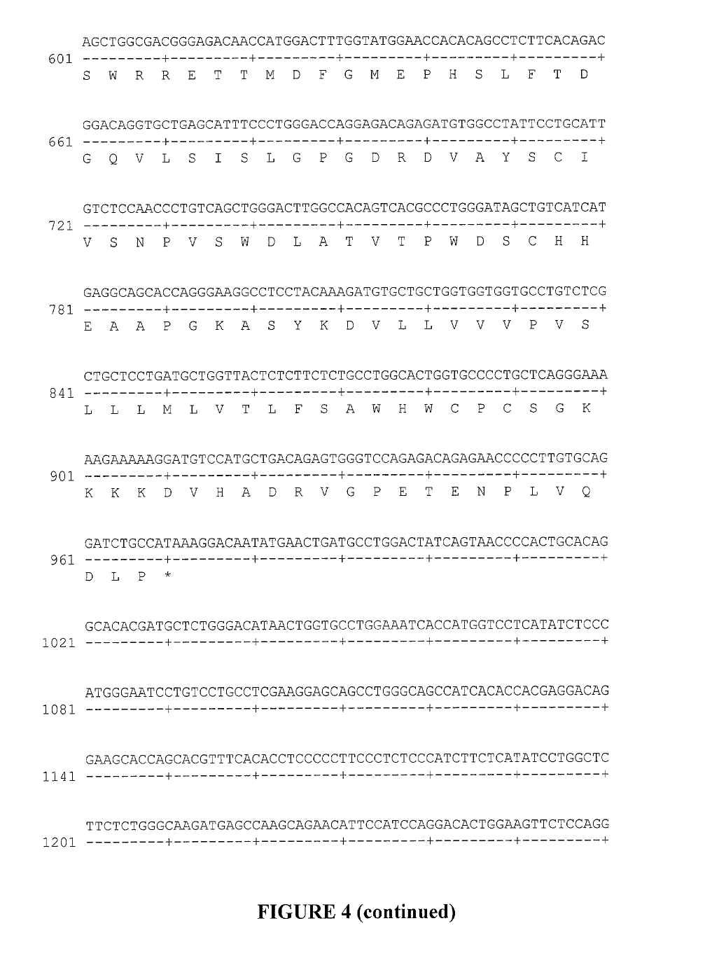

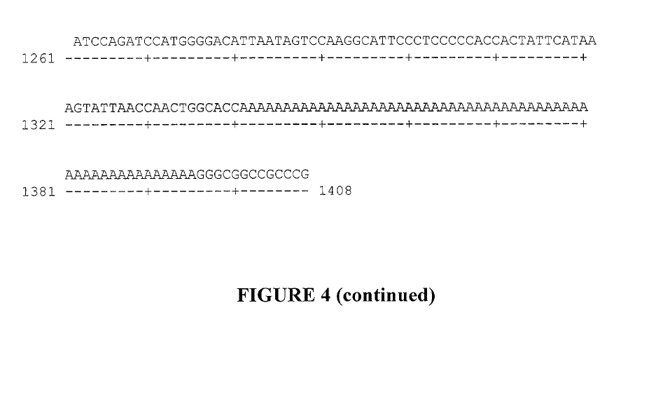

In another embodiment, apex-3 sequence as shown in SEQ ID NO. 3, comprises the coding sequence of APEX-3 protein between nucleotides 115 and 972. The 868 base pair coding region of apex-3 beginning at adenine (a) at position 115 and ending at adenine (a) at position 972 as shown in SEQ ID NO. 3 can encode a 285 amino acid APEX-3 protein. The APEX-3 protein is comprised of a signal peptide, an extracellular domain, a transmembrane domain, and a cytoplasmic domain (FIG. 4).

The nucleic acid molecules of the invention may be recombinant DNA molecules each comprising the sequence of apex -1, -2 or -3 (or fragments or derivatives thereof) fused to non-apex sequences, such as human Ig or FLAG: DYKDDDDK (Sigma-Aldrich Corporation, St. Louis, Mo.), which is a commercially available 8 amino acid sequence tag. Reviews of methods for synthesis of oligonucleotides can be found in: Oligonucleotides and Analogues, eds. F. Eckstein, 1991, IRL Press, New York; Oligonucleotide Synthesis, ed. M. J. Gait, 1984, IRL Press, Oxford, England.

The present invention also provides uses of the apex nucleotide sequences and their corresponding amino acid sequences, and antibodies reactive against the APEX proteins for the study, diagnosis, prevention and treatment of disease associated with the presence of an APEX protein.

Fragments of Apex

Apex e.g., apex-1, -2, and -3, includes fragments can be used as selective hybridization probes or PCR primers. These fragments can be readily identified from the entire sequence of the APEX proteins, using art-known methods. A “primer” is a nucleic acid fragment which functions as an initiating substrate for enzymatic or synthetic elongation of, for example, a nucleic acid molecule. For example, sets of PCR primers that are useful for detecting transcripts encoding the extracellular domain of an APEX protein comprise the forward primer JNF6 (5′-atc ctt tgg cag ctc aca gg-3′; SEQ ID NO.: 12) and the reverse primer JNF7 (5′-ctt cac aga gct tcc tgg c-3′; SEQ ID NO.: 13).

Complementary Sequences

The nucleic acid molecules provided by the present invention include DNA molecules each comprising the nucleotide sequence, or portions thereof, which are complementary to the nucleotide sequences as described in e.g. SEQ ID NO.: 1, 2, or 3.

The term “complementary” as used herein refers to the capacity of purine and pyrimidine nucleotides to associate through hydrogen bonding to form double stranded nucleic acid molecules. The following base pairs are related by complementarity: guanine and cytosine; adenine and thymine; and adenine and uracil. Complementary applies to all base pairs comprising two single-stranded nucleic acid molecules.

The present invention also provides complementary nucleic acid molecules having various degrees of sequence similarity with the apex sequences, which are exactly complementary to SEQ ID NO. 1, SEQ ID NO.2, or SEQ ID NO. 3. For example, nucleotide sequences that are substantially similar to the exact complementary apex sequences or portions thereof, will hybridize to an apex-1, -2 or -3 sequence under high stringency hybridization conditions. Typically, hybridization under standard high stringency conditions will occur between two nucleic acid molecules that differ in sequence by about 80% to about 99%. It is readily apparent to one skilled in the art that the high stringency hybridization between nucleic acid molecules depends upon, for example, the degree of similarity, the stringency of hybridization, and the length of hybridizing strands. The methods and formulas for conducting high stringency hybridizations are well known in the art, and can be found in, for example, Sambrook et al, Molecular Cloning (1989).

Allelic Forms of Apex

The present invention provides nucleotide sequences of cDNA encoding allelic forms of apex. For example, one allelic form of human apex-1 is described in FIG. 2 (SEQ ID NO.: 1). The nucleotide sequence of a cDNA encoding one allelic form of murine apex-2 is described in FIG. 3 (SEQ ID NO.: 2), and the nucleotide sequence of a cDNA encoding one allelic form of human apex-3 is described in FIG. 4 (SEQ ID NO.: 3) As used herein, an “allele” or “allelic sequence” is an alternative form of the apex gene. Alleles result from a mutation, such as, a change in the nucleotide sequence, and generally produce altered mRNAs or polypeptides whose structure or function may or may not be altered. The present invention contemplates other allelic forms of nucleic acid molecules encoding apex-1, -2 and -3 that are isolated from different subjects of the same species. Identification of allelic variants is known in the art.

Homologues

The present invention provides nucleotide sequences that encode APEX homologues. For example, the invention provides the human homologue of APEX-1 and -3, and the murine homologue of APEX-2. One embodiment of the invention also provides the nucleotide sequences of apex-1, -2, and -3 homologues isolated from other species. Protein homologues from different species are related as a result of common ancestry. The ancestral homologue may have undergone speciation (e.g., an ortholog) or gene duplication (e.g., a paralog). Thus, protein homologues are typically isolated from different species and have the same or similar function. As a result of the common ancestry, homologues may or may not have similar amino acid sequences. The homologues can be from any species particularly mammalian, including bovine, ovine, porcine, murine, equine, and preferably human. Methods for the identification of APEX homologues are routine and well known in the art (Sambrook et al., Molecular Cloning (1989).

Variant Nucleotide Sequences

It may be advantageous to generate codon-usage variants that are altered from the disclosed apex nucleotide sequences, yet do not alter the amino acid sequence of the encoded APEX proteins. The codons may be selected to optimize the level of production of the apex transcript or APEX protein in a particular prokaryotic or eukaryotic expression host, in accordance with the frequency of codon utilized by the host cell. Alternative reasons for altering the nucleotide sequence encoding an APEX protein include the production of RNA transcripts having more desirable properties, such as an increased half-life. A multitude of variant apex nucleotide sequences that encode the respective APEX proteins may be isolated, as a result of the degeneracy of the genetic code. Accordingly, the present invention contemplates selecting every possible triplet codon to produce every possible combination of nucleotide sequences that encode the disclosed amino acid sequence of APEX-1, -2 or -3 proteins. One embodiment of the present invention provides isolated nucleotide sequences that vary from the sequences as described in SEQ ID NO.: 1, 2, or 3, such that each variant nucleotide sequence encodes a polypeptide having sequence identity with the amino acid sequence of APEX-1, -2, or -3, as described in SEQ ID NO.: 4, 5, or 6, respectively.

RNA

The present invention provides RNA molecules that encode APEX proteins. In particular, the RNA molecules of the invention may be isolated full-length or partial mRNA molecules, or RNA oligomers that encode APEX-1, -2, or -3. The RNA molecules of the invention each include the nucleotide sequences encoding all or portions of the signal peptide region, the extracellular domain, the transmembrane domain, and/or the intracellular domain of APEX -1, -2 or -3.

The RNA molecules of the invention also include antisense RNA molecules, peptide nucleic acids (PNAs), or non-nucleic acid molecules such as phosphorothioate derivatives, that specifically bind to the sense strand of DNA or RNA in a base pair-dependent manner. A skilled artisan can readily obtain these classes of nucleic acid molecules using the herein described apex sequences.

Peptide Nucleic Acids

The nucleic acid molecules of the invention include peptide nucleic acids (PNAs), or derivative molecules such as phosphorothioate, phosphotriester, phosphoramidate, and methylphosphonate, that specifically bind to single-stranded apex DNA or RNA in a base pair-dependent manner (P. C. Zamecnik, et al., 1978 Proc. Natl. Acad. Sci. 75:280284; Goodchild, P. C., et al., 1986 Proc. Natl. Acad. Sci. 83:4143-4146). A skilled artisan can readily obtain these classes of nucleic acid molecules using the herein described apex sequences. For example, reviews of methods for synthesis of DNA, RNA, and their analogues can be found in: Oligonucleotides and Analogues, eds. F. Eckstein, 1991, IRL Press, New York; Oligonucleotide Synthesis, ed. M. J. Gait, 1984, IRL Press, Oxford, England. Additionally, methods for antisense RNA technology are described in U.S. Pat. Nos. 5,194,428 and 5,110,802.

A PNA molecule comprises a nucleic acid oligomer to which an amino acid residue, such as lysine, and an amino group have been added. These small molecules, also designated anti-gene agents stop transcription elongation by binding to the complementary strand of nucleic acid (Nielsen, P. E. et al. 1993 Anticancer Drug Des, 8:53-63). A skilled artisan can readily obtain these classes of nucleic acid molecules using the herein described modified apex nucleotide sequences, see for example Innovative and Perspectives in Solid Phase Synthesis (1992) Egholm, et al. pp 325-328 or U.S. Pat. No. 5,539,082.

Nucleic Acid Molecules Labeled with A Detectable Marker

Embodiments of the APEX-encoding nucleic acid molecules of the invention include DNA and RNA primers, which allow the specific amplification of nucleic acid molecules of the invention or of any specific parts thereof, and probes that selectively or specifically hybridize to nucleic acid molecules of the invention or to any part thereof. The nucleic acid probes can be labeled with a detectable marker. Examples of a detectable marker include, but are not limited to, a radioisotope, a fluorescent compound, a bioluminescent compound, a chemiluminescent compound, a metal chelator or an enzyme. Technologies for generating labeled DNA and RNA probes are well known.

Apex Proteins and Polypeptides

APEX proteins of this invention belong to the Ig superfamily and may be involved in ligand binding and signal transduction. The predicted sequence of APEX proteins includes a N-terminal hydrophobic signal peptide, an extracellular domain consisting of two Ig-like regions, a transmembrane domain, and an intracellular domain (for description, See Examples 1-3). APEX proteins can be full length or fragments thereof that have APEX activity.

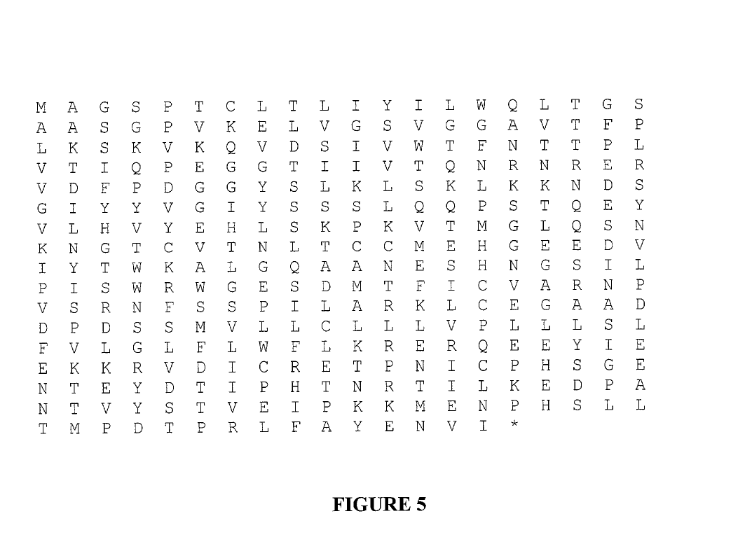

In one embodiment, the APEX-1 protein includes a N-terminal 22 amino acid hydrophobic signal peptide, a 203 amino acid extracellular domain, a 24 amino acid transmembrane domain, and an 86 amino acid intracellular domain (see FIG. 5). APEX-1 is encoded by nucleotide sequence 42 to 1049 as shown in SEQ ID NO. 1. Further, APEX-1 includes an extracellular domain. The extracellular domain of APEX-1 can be encoded by, for example, nucleotide sequence 108-716 as shown in SEQ ID NO. 1.

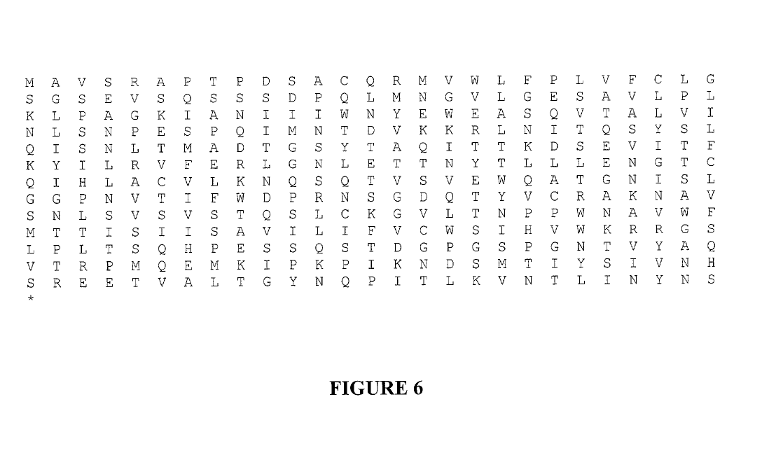

In another embodiment, APEX-2 protein comprises a putative signal peptide of 29 amino acids, a 210 amino acid extracellular domain, a 23 amino acid transmembrane domain, and an 89 amino acid cytoplasmic domain (see FIG. 6). APEX-2 is encoded by nucleotide sequence 162 to 1217 as shown in SEQ ID NO. 2. Further, APEX-2 includes an extracellular domain. The extracellular domain of APEX-2 can be encoded by, for example, nucleotide sequence 249 to 875 as shown in SEQ ID NO. 2.

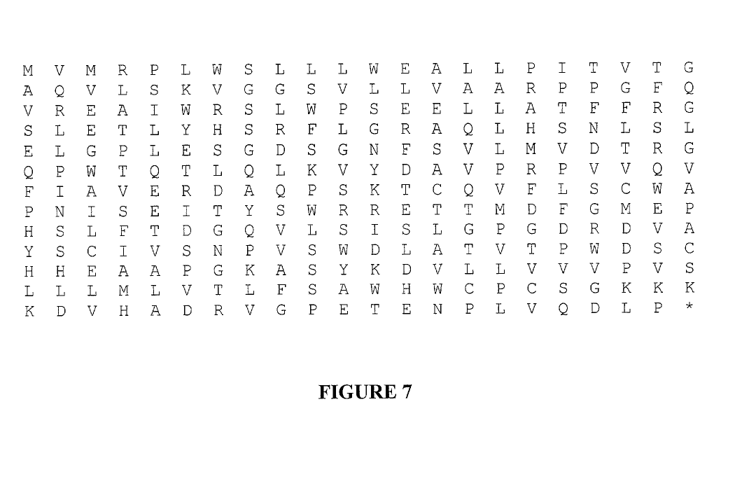

In yet another embodiment, APEX-3 comprises a 22 amino acid signal peptide, a 209 amino acid extracellular domain, a 23 amino acid transmembrane domain, and a 31 amino acid cytoplasmic domain (FIG. 7). The putative cytoplasmic domain of APEX-3 is much shorter than the cytoplasm of other CD2 subgroup members. APEX-3 is encoded by, for example, nucleotide sequence 115 to 972 as shown in SEQ ID NO. 3.

APEX proteins share structural similarities with members of the CD2 subfamily. Thus, it is postulated that APEX proteins represent a family of cell-surface receptors that regulate adhesion and generate co-stimulatory signals to mediate leukocyte proliferation, differentiation, migration, or activation. It is possible that APEX proteins enhance antigen-specific proliferation and cytokine production, similar to SLAM which is another member of the CD2 subfamily. Thus, APEX proteins may prove to be a potential target for diseases with an inflammatory and autoimmune component.

As used herein, APEX proteins of this invention include a protein that has the amino acid sequence of human APEX-1; as provided in FIG. 5 (e.g., SEQ ID NO.: 4), the amino acid sequence of murine APEX-2 as provided in FIG. 6 (e.g., SEQ ID NO.: 5), the amino acid sequence of human APEX-3 as provided in FIG. 7 (SEQ ID NO.: 6), or other mammalian APEX homologues, as well as allelic variants and conservative substitution mutants of these proteins, that have structural similarities to APEX proteins of the invention, or APEX activity or function. One aspect of the invention provides various APEX proteins and peptide fragments thereof having APEX activity. For the sake of convenience, all APEX proteins will be collectively referred to as the APEX proteins, the proteins of the invention, or APEX.

As used herein, “APEX” refers to the amino acid sequence of APEX polypeptides from any species, particularly mammalian, including bovine, ovine, porcine, murine, equine, and preferably human, in a naturally occurring form or from any source whether natural, synthetic, semi-synthetic or recombinant. As used herein, “naturally occurring” refers to an amino acid sequence which is found in nature.

The term “having APEX activity” refers to an APEX protein or fragment having a function of the naturally occurring APEX and/or ability to recognize and bind ligands or antibodies directed against APEX. The term “APEX activity” further defines the capability to induce a specific immune response in appropriate animals or cells and to bind with specific APEX antibodies.

The term “derivative” as used herein refers to a chemical modification of the apex nucleic acid molecule or the encoded APEX protein. Illustrative of such modifications of an APEX protein would be replacement of hydrogen by an alkyl, acyl, or amino group. An APEX derivative would encode a polypeptide which retains the essential biological characteristics and activities of natural APEX.

Variant Polypeptides and Proteins

The present invention provides APEX proteins including all isolated, naturally occurring or recombinantly made allelic variants, isoforms, and precursors of human APEX-1 or -3 as provided in FIGS. 5 and 7, respectively; and murine APEX-2 as provided in FIG. 5. In general, for example, naturally occurring allelic variants of human or murine APEX will share significant homology (e.g., about 70-99%) to the APEX amino acid sequences provided in FIGS. 5, 6, and 7. Allelic variants, though possessing a slightly different amino acid sequence, may be expressed on the surface of APCs cells or may be secreted or shed.

Typically, allelic variants of the APEX protein can include one or more conservative amino acid substitutions from the APEX sequence herein described or will include a substitution of an amino acid from a corresponding position in an APEX homologue such as, for example, the murine APEX homologue described herein.

One type of allelic variant of apex encodes APEX proteins having amino acid sequences with one or more amino acid substitutions, insertions, deletions, truncations, or frame shifts. Such alleles are termed mutant alleles of APEX and represent proteins that may or may not perform the same biological functions as wild-type APEX, such as function as a cell-surface receptor.

Another variant of APEX may have an amino acid sequence that is different by one or more amino acid “substitutions”. The variant may have “conservative” changes, wherein a substituted amino acid has similar structural or chemical properties, e.g., replacement of leucine with isoleucine. Alternatively, a variant may have “nonconservative” changes, e.g., replacement of a glycine with a tryptophan. Similar minor variations may also include amino acid deletions or insertions, or both. Guidance in determining which and how many amino acid residues may be substituted, inserted or deleted may be found using computer programs well known in the art, for example, DNASTAR software.

Conservative amino acid substitutions can frequently be made in a protein without altering either the conformation or the function of the protein. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments. APEX proteins may be embodied in many forms, preferably in isolated form. As used herein, a protein is said to be isolated when physical, mechanical or chemical methods are employed to remove the APEX protein from cellular constituents that are normally associated with the protein. A skilled artisan can readily employ standard purification methods to obtain an isolated APEX protein, see for example “Strategies for Protein Purification and Characterization ” (1996) pp 396, Marshak, D. R. et al.

A purified APEX protein molecule will be substantially free of other proteins or molecules that impair the binding of APEX to antibody or other ligand. The nature and degree of isolation and purification will depend on the intended use. Embodiments of the APEX protein include a purified APEX protein, or fragment thereof having APEX activity. Examples of a purified APEX protein include proteins having the amino acid sequence shown in FIGS. 5, 6, or 7, or a fragment thereof. In one form, such purified APEX proteins, or fragments thereof, retain the ability to bind antibody or other ligand.

The term “purified” as used herein means a specific isolated nucleic acid molecule or protein, or fragment thereof, in which substantially all contaminants (i.e. substances that differ from said specific molecule) have been separated from said nucleic acid molecule or protein. For example, a protein may, but not necessarily, be “substantially purified” by the immuno affinity column chromatography (IMAC) method.

Peptides

APEX proteins and APEX proteins and peptide fragments of APEX having APEX activity can be generated using standard peptide synthesis technology and the amino acid sequences of the human or murine APEX proteins disclosed herein. The principles of solid phase chemical synthesis of polypeptides are well known in the art and may be found in general texts relating to this area (H. Dugas and C. Penney, 1981 in: “Bioorganic Chemistry, ”Springer-Verlag, New York, pp 54-92). The polypeptides of the invention may be synthesized by solid-phase methodology utilizing an Applied Biosystems 430A peptide synthesizer (Applied Biosystems, Foster City, Calif.) and synthesis cycles supplied by Applied Biosystems. Protected amino acids, such as t-butoxycarbonyl-protected amino acids, and other reagents are commercially available from many chemical supply houses.

Alternatively, recombinant methods can be used to generate nucleic acid molecules that encode a fragment of the APEX protein having the sequence and/or activity of APEX protein. In this regard, the APEX-encoding nucleic acid molecules described herein provide means for generating defined fragments of APEX protein.

As discussed below, peptide fragments of APEX are particularly useful in: generating domain specific antibodies; identifying agents that bind to APEX or an APEX domain; identifying cellular factors that bind to APEX or an APEX domain; and isolating homologues or other allelic forms of APEX. APEX peptides, including particularly interesting structures, can be predicted and/or identified using various analytical techniques well known in the art (Rost, B., and Sander, C. 1994 Proteins 19:55-72), including, for example, the methods of Chou-Fasman, Garnier-Robson, Kyte-Doolittle, Eisenberg, Karplus-Schultz or Jameson-Wolf analysis, or on the basis of immunogenicity. Fragments including such residues are particularly useful in generating subunit-specific anti-APEX antibodies or in identifying cellular factors that bind to APEX.

The APEX proteins of the invention may be useful for a variety of purposes, including but not limited to their use as diagnostic and/or prognostic markers on APCs or APEX-expressing cells, the ability to elicit the generation of antibodies, and as targets for various therapeutic modalities, as further described below. APEX proteins may also be used to identify and isolate ligands and other agents that bind to APEX.

Apex Antibodies and Uses Thereof

Antibodies

The invention further provides antibodies (e.g., polyclonal, monoclonal, chimeric, humanized, and human monoclonal antibodies) that bind to APEX. The most preferred antibodies will selectively bind to APEX and will not bind (or will bind weakly) to non-APEX proteins. Anti-APEX antibodies that are particularly contemplated include monoclonal and polyclonal antibodies as well as fragments thereof (e.g., recombinant proteins) including the APEX antigen-binding domain and/or one or more complement determining regions of these antibodies. These antibodies can be from any source, e.g., rabbit, sheep, rat, dog, cat, pig, horse, mouse and human.

In one embodiment, the APEX antibodies specifically bind to the extracellular domain of an APEX protein. In other embodiments, the APEX antibodies specifically bind to other domains of an APEX protein or precursor, for example the APEX antibodies bind to the intracellular domain. As will be understood by those skilled in the art, the regions or epitopes of an APEX protein to which an antibody is directed may vary with the intended application. For example, antibodies intended for use in an immunoassay for the detection of membrane-bound APEX on viable cells should be directed to an accessible epitope on membrane-bound APEX. Antibodies that recognize other epitopes may be useful for the identification of APEX within damaged or dying cells, for the detection of secreted APEX proteins or fragments thereof.

The invention also encompasses antibody fragments that specifically recognize an APEX protein. As used herein, an antibody fragment is defined as at least a portion of the variable region of the immunoglobulin molecule that binds to its target, i.e., the antigen binding region. Some of the constant region of the immunoglobulin may be included.

For example, the predicted extracellular domain of APEX represents characteristics of a potential marker for screening, diagnosis, prognosis, and follow-up assays and imaging methods. In addition, these characteristics indicate that APEX may be an excellent target for therapeutic methods such as targeted antibody therapy, immunotherapy, and gene therapy to treat conditions associated with the presence or absence of APEX proteins.

Various methods for the preparation of antibodies are well known in the art. For example, antibodies may be prepared by immunizing a suitable mammalian host using an APEX protein, peptide, or fragment, in isolated or immunoconjugated form (Harlow (1989), in: Antibodies, Cold Spring Harbor Press, NY). In addition, fusion proteins of APEX may also be used, such as an APEX-GST, or APEX-tagged fusion proteins, or APEX-human Ig—for example Human Ig and other mammalian species especially mouse. Cells expressing or overexpressing APEX may also be used for immunizations. Similarly, any cell engineered to express APEX may be used. This strategy may result in the production of monoclonal antibodies with enhanced capacities for recognizing endogenous APEX.

Chimeric antibodies of the invention are immunoglobulin molecules that comprise at least two antibody portions from different species, for example a human and non-human portion. The antigen combining region (variable region) of a chimeric antibody can be derived from a non-human source (e.g. murine), and the constant region of the chimeric antibody which confers biological effector function to the immunoglobulin, can be derived from a human source. The chimeric antibody should have the antigen binding specificity of the non-human antibody molecule and the effector function conferred by the human antibody molecule.

Antibodies of several distinct antigen binding specificities have been manipulated to produce chimeric proteins such as anti-TNP (Boulianne et al., Nature 312:643 (1984)) and anti-tumor antigens (Sahagan et al., J. Immunol. 137:1066 (1986)). Likewise, several different effector functions have been achieved by linking new sequences to those encoding the antigen binding region. Some of these include enzymes (Neuberger et al., Nature 312:604 (1984)), immunoglobulin constant regions from another species and constant regions of another immunoglobulin chain (Sharon et al., Nature 309:364 (1984); Tan et al., J. Immunol. 135:3565-3567 (1985)). Additionally, procedures for modifying antibody molecules and for producing chimeric antibody molecules using homologous recombination to target gene modification have been described (Fell et al., Proc. Natl. Acad. Sci. USA 86:8507-8511 (1989)).

APEX mAbs can be used to stain the cell surface of APEX positive cells for detection. Additionally, some of the antibodies of the invention are internalizing antibodies, i.e., the antibodies are internalized into the cell upon or after binding.

The amino acid sequences of APEX proteins presented herein may be used to select specific regions of the APEX protein for generating antibodies. For example, hydrophobicity or hydrophilicity analyses of the APEX amino acid sequence may be used to identify hydrophilic regions in the APEX structure. Regions of the APEX protein that show immunogenic structure, as well as other regions and domains, can readily be identified using various other methods known in the art, such as Chou-Fasman, Gamier-Robson, Kyte-Doolittle, Eisenberg, Karplus-Schultz or Jameson-Wolf analysis. Fragments including these residues are particularly suited for generating specific classes of anti-APEX antibodies.

Methods for preparing a protein for use as an immunogen and for preparing immunogenic conjugates of a protein with a carrier such as BSA, KLH, or other carrier proteins are well known in the art (Harlow and Lane, 1988, in: Antibodies: A Laboratory Manual. Cold Spring Harbor Press). In some circumstances, direct conjugation using, for example, carbodiimide reagents may be employed; in other instances linking reagents such as those supplied by Pierce Chemical Co., Rockford, Ill., may be effective. Administration of an APEX immunogen is conducted generally by injection over a suitable time period and with use of a suitable adjuvant, as is generally understood in the art. During the immunization schedule, titers of antibodies can be taken to determine adequacy of antibody formation.

While the polyclonal antisera produced in this way may be satisfactory for some applications, for pharmaceutical compositions, monoclonal antibody preparations are preferred. Immortalized cell lines which secrete a desired monoclonal antibody may be prepared using the standard method of Kohler and Milstein (Nature 256: 495-497) or modifications which effect immortalization of lymphocytes or spleen cells, as are generally known. The immortalized cell lines secreting the desired antibodies are screened by immunoassay in which the antigen is the APEX protein or APEX fragment having APEX activity. When the appropriate immortalized cell culture secreting the desired antibody is identified, the cells can be cultured either in vitro, or by production in ascites fluid.

The desired monoclonal antibodies are then recovered from the culture supernatant or from the ascites supernatant. Fragments of the monoclonal antibodies of the invention or the polyclonal antisera (e.g., Fab, F(ab′)₂, Fv fragments, fusion proteins), which include the immunologically significant portion (i.e., a portion that recognizes and binds APEX), can be used as antagonists, as well as the intact antibodies.

Humanized antibodies directed against APEX are also useful. As used herein, a humanized APEX antibody is an immunoglobulin molecule which is capable of binding to APEX, and which comprises a framework region (FR) region having substantially the amino acid sequence of a human immunoglobulin, and a complementarity determining region (CDR) having substantially the amino acid sequence of non-human immunoglobulin, or a sequence engineered to bind APEX. These humanized or “chimeric antibodies” are produced by splicing of mouse antibody genes to human antibody genes to obtain a molecule with appropriate antigen specificity and biological activity (Morrison, S. L. et al. (1984) Proc. Natl. Acad. Sci. 81, 6851-6855; Neuberger, M. S. et al. (1984) Nature 312, 604-608; and Takeda, S. et al. (1985) 314, 452-454). Alternatively, single chain antibodies may be developed using methods known in the art, to produce APEX-specific single chain antibodies. Antibodies with related specificity, but of distinct idiotypic composition, may also be produced by chain shuffling from random combinatorial immunoglobulin libraries (Burton, D. R. (1991) Proc. Natl. Acad. Sci. 86, 3833-3837; Winter, G. et al. (1991) Nature 349, 293-299).

Use of immunologically reactive fragments, such as the Fab, Fab′, or F(ab′)₂ fragments is often preferable, especially in a therapeutic context, as these fragments are generally less immunogenic than the whole immunoglobulin. The invention also provides pharmaceutical compositions having the monoclonal antibodies or anti-idiotypic monoclonal antibodies of the invention.

The antibodies or fragments may also be produced, using current technology, by recombinant means. Regions that bind specifically to the desired regions of the APEX protein can also be produced in the context of chimeric or CDR grafted antibodies of multiple species origin. The invention includes a monoclonal antibody, the antigen-binding region of which competitively inhibits the immunospecific binding of any of the monoclonal antibodies of the invention to its target antigen. Further, the invention provides recombinant proteins comprising the antigen-binding region of any the monoclonal antibodies of the invention.

Novel antibodies of human origin can be also made to the antigen having the appropriate biological functions. The completely human antibodies are particularly desirable for therapeutic treatment of human patients. The human monoclonal antibodies may be made by using the antigen, e.g. an APEX protein or peptide thereof, to sensitize human lymphocytes to the antigen in vitro, followed by EBV-transformation or hybridization of the antigen-sensitized lymphocytes with mouse or human lymphocytes, as described by Borrebaeck et al. (Proc. Natl. Acad. Sci. USA 85:3995-99 (1988)).

Alternatively, human antibodies can be produced using transgenic animals such as mice which are incapable of expressing endogenous immunoglobulin heavy and light chain genes, but which can express human heavy and light chain genes. The transgenic mice are immunized in the normal fashion with a selected antigen, e.g., all or a portion of a polypeptide of invention. Monoclonal antibodies directed against the antigen can be produced using conventional hybridoma technology. The human immunoglobulin transgenes harbored by the transgenic mice rearrange during B cell differentiation, and subsequently undergo class switching and somatic mutations. Thus, using this technology, it is possible to produce therapeutically useful IgG, IgA, and IgB antibodies. For an overview of this technology to produce human antibodies, see Lonberg and Haszar (1995, Int. Rev. Immunol. 13;65-93). A detailed discussion of this technology for producing human antibodies and human monoclonal antibodies can be found in U.S. Pat. Nos. 5,625,126; 5,633,425; 5,569,825; 5,661,016; and 5,545,806.

The antibody, or fragment thereof, of the invention may be labeled with a detectable marker or conjugated to a second molecule, such as a therapeutic agent (e.g., a cytotoxic agent), thereby resulting in an immunoconjugate. For example, the therapeutic agent includes, but is not limited to, an anti-tumor drug, a toxin, a radioactive agent, a cytokine, a second antibody or an enzyme. Further, the invention provides an embodiment wherein the antibody of the invention is linked to an enzyme that converts a prodrug into a cytotoxic drug. The immunoconjugate can be used for targeting the second molecule to an APEX positive cell (Vitetta, E. S. et al., 1993 “Immunotoxin Therapy”, in DeVita, Jr., V. T. et al., eds, Cancer. Principles and Practice of Oncology, 4th ed., J. B. Lippincott Co., Philadelphia, 2624-2636).

Examples of cytotoxic agents include, but are not limited to ricin, doxorubicin, daunorubicin, taxol, ethiduim bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme.

Additionally, the recombinant protein of the invention comprising the antigen-binding region of any of the monoclonal antibodies of the invention, can be used to treat diseases associated with the presence of APEX proteins. In such a situation, the antigen-binding region of the recombinant protein is joined to at least a functionally active portion of a second protein having therapeutic activity. The second protein can include, but is not limited to, an enzyme, lymphokine, oncostatin or toxin. Suitable toxins include doxorubicin, daunorubicin, taxol, ethiduim bromide, mitomycin, etoposide, tenoposide, vincristine, vinblastine, colchicine, dihydroxy anthracin dione, actinomycin D, diphteria toxin, Pseudomonas exotoxin (PE) A, PE40, ricin, abrin, glucocorticoid and radioisotopes.

Techniques for conjugating or joining therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev. 62:119-58 (1982)).

Use of the Antibodies

APEX antibodies of the invention may be particularly useful in diagnostic assays and imaging methodologies. The invention provides various immunological assays useful for the detection of APEX proteins. Such assays generally comprise one or more APEX antibodies capable of recognizing and binding an APEX protein, and include various immunological assay formats well known in the art, including but not limited to various types of precipitation, agglutination, complement fixation, radioimmunoassays (RIA), enzyme-linked immunosorbent assays (ELISA), enzyme-linked immunofluorescent assays (ELIFA) (H. Liu et al. Cancer Research 58: 4055-4060 (1998), immunohistochemical analysis and the like.

In order to provide a basis for diagnosis, normal or standard values for APEX expression must be established. This is accomplished by combining body fluids or cell extracts taken from normal subjects with antibody to APEX under conditions suitable for complex formation, which are well known in the art. The amount of standard complex formation may be quantified by comparing various artificial membranes containing known quantities of APEX with both control and disease samples from biopsied tissues. Then, standard values obtained from normal samples may be compared with values obtained from subjects potentially affected by disease. Deviation between standard and subject values establishes the presence of a disease state.

In addition, immunological imaging methods capable of detecting APEX-expressing cells are also provided by the invention, including, but not limited to, radioscintigraphic imaging methods using labeled APEX antibodies. Such assays may be clinically useful in the detection and monitoring.

In one embodiment, APEX antibodies and fragments thereof (e.g., Fv, Fab′, F(ab′)2) are used for detecting the presence of a cell expressing an APEX protein. The presence of such APEX positive (+) cells within various biological samples, including blood, serum, urine, tissue, etc., may be detected with APEX antibodies. In addition, APEX antibodies may be used in various imaging methodologies, such as immunoscintigraphy with Induim-111 (or other isotope) conjugated antibody (Sodee et al., 1997, Clin Nuc Med 21: 759-766). APEX antibodies may also be used therapeutically to inhibit APEX function.

APEX antibodies may be used in methods for purifying APEX proteins and peptides and for isolating APEX homologues and related molecules. For example, in one embodiment, the method of purifying APEX protein comprises incubating an APEX antibody, which has been coupled to a solid matrix, with a lysate or other solution including APEX under conditions which permit the APEX antibody to bind to APEX; washing the solid matrix to eliminate impurities; and eluting the APEX from the coupled antibody. Additionally, APEX antibodies may be used to isolate APEX positive cells using cell sorting and purification techniques. The presence of APEX on cells (alone or in combination with other cell surface markers) may be used to distinguish and isolate APEX-expressing cells from other cells, using antibody-based cell sorting or affinity purification techniques. Other uses of the APEX antibodies of the invention include generating anti-idiotypic antibodies that mimic the APEX protein, e.g., a monoclonal anti-idiotypic antibody reactive with an idiotype on any of the monoclonal antibodies of the invention.

The antibodies of the invention may be used to generate large quantities of relatively pure APEX-positive cells which can be grown in tissue culture or administered ex vivo to a subject.

Another valuable application of using antibodies to generate APEX-positive cells is the ability to isolate, analyze and experiment with relatively pure preparations of viable APEX positive cells cloned from individual subjects or patients. In this way, for example, an individual subject's cells may be expanded from a limited biopsy sample and then tested for the presence of diagnostic and prognostic genes, proteins, chromosomal aberrations, gene expression profiles, or other relevant genotypic and phenotypic characteristics, without the potentially confounding variable of contaminating cells. Similarly, patient-specific vaccines and cellular immunotherapeutics may be created from such cell preparations.

Other uses of the APEX antibodies of the invention include generating anti-idiotypic antibodies that mimic the APEX proteins of the invention, e.g., a monoclonal anti-idiotypic antibody reactive with an idiotype on any of the monoclonal antibodies of the invention. Anti-idiotypic antibodies of the APEX antibody may be used therapeutically in the treatment of APEX-associated disorders.

Methods for Isolating Additional Apex-encoding Nucleic Acid Molecules

The APEX-encoding nucleic acid molecules described herein enable the isolation of APEX homologues, alternatively sliced isoforms, allelic variants, and mutant forms of the APEX protein as well as their coding and gene sequences. The most preferred sources of APEX homologues are mammalian organisms.

For example, a portion of the APEX-encoding sequence herein described can be synthesized and used as a probe to retrieve DNA encoding a member of the APEX family of proteins from organisms other than human, allelic variants of the human APEX protein herein described, and genomic sequence including the apex gene. Oligomers containing approximately 18-20 nucleotides (encoding about a 6-7 amino acid stretch) are prepared and used to screen genomic DNA or cDNA libraries to obtain hybridization under stringent conditions, or conditions of sufficient stringency, to eliminate an undue level of false positives.

In addition, the amino acid sequence of the human or murine APEX protein may be used to generate antibody probes to screen expression libraries prepared from cells to obtain APEX homologues from other mammalian species. Typically, polyclonal antiserum from mammals such as rabbits immunized with the purified protein or monoclonal antibodies can be used to probe an expression library prepared from a target organism, to obtain the appropriate coding sequence for an APEX homologue. The cloned cDNA sequence can be expressed as a fusion protein, expressed directly using its own control sequences, or expressed by constructing an expression cassette using control sequences appropriate to the particular host used for expression of the enzyme.

Genomic clones including APEX genes may be obtained using molecular cloning methods well known in the art. In one embodiment, an apex cDNA probe can be used to screen a genomic library, such as libraries constructed in lambda phage, or BAC (bacterial artificial chromosome) or YAC (yeast artificial chromosome), to obtain a genomic clone of an apex gene.

Additionally, pairs of oligonucleotide primers can be prepared for use in a polymerase chain reaction (PCR) to selectively amplify/clone an APEX-encoding nucleic acid molecule, or fragment thereof. A PCR denature/anneal/extend cycle for using such PCR primers is well known in the art (U.S. Pat. No. 4,683,202 and 4,965,188) and can readily be adapted for use in isolating other APEX-encoding nucleic acid molecules.

Non-human homologues of apex, naturally occurring allelic variants of apex and genomic apex sequences will share a high degree of homology to the human apex sequences herein described. In general, such nucleic acid molecules will hybridize to the human apex sequence under stringent conditions. Such sequences will typically have at least 70% homology, preferably at least 80%, and most preferably at least 90% homology to the human apex sequence.

Stringent conditions are those that employ low ionic strength and high temperature for washing. For example, stringent salt concentration will ordinarily be less than 750 mM NaCl and 75 mM sodium citrate, preferably less than about 500 mM NaCl and 50 mM sodium citrate, and most preferably less than about 250 mM NaCl and 25 mM sodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will normally include a temperature of at least about 30° C., more preferably at least about 37° C., and most preferably of at least about 42° C. Varying additional parameters, such as hybridization time, the concentration of detergent, e.g., sodium dodecyl sulfate (SDS), and the inclusion or exclusion of carrier DNA are well known to those skilled in the art. Various levels of stringency are achieved by combining these various conditions as needed. In a preferred embodiment, hybridization will occur at 30° C. in 750 mM NaCl, 75 mM sodium citrate, and 1% SDS. In a more preferred embodiment, hybridization will occur at 37° C. in 500 mM NaCl, 50 mM sodium citrate, 1% SDS, 35% formamide, and 100 μg/ml denatured salmon sperm DNA (ssDNA). In a most preferred embodiment, hybridization will occur at 42° C. in 250 mM NaCl, 25 mM sodium citrate, 1% SDS, 50% formamide, and 200 μg/ml ssDNA. Appropriate variations on these conditions will be readily apparent to those skilled in the art.

The washing steps that follow hybridization can also vary in stringency. Wash stringency conditions can be defined by salt concentration and by temperature. As with the hybridization, wash stringency can be increased by decreasing salt concentration or by increasing temperature. For example, stringent salt concentration for the wash steps will preferably be less than about 30 mM NaCl and 3 mM sodium citrate, and most preferably, less than about 15 mM NaCl and 1.5 mM sodium citrate. Stringent temperature conditions for wash steps will normally include a temperature of at least 25° C., more preferably of at least 42° C., and most preferably of at least about 68° C. In a preferred embodiment, wash steps will occur at 25° C. in 30 mM NaCl, 3 mM sodium citrate, and 0.1% SDS. In a more preferred embodiment, wash steps will occur at 42° C. in 15 mM NaCl, 1.5 mM sodium citrate and 0.1% SDS. In a most preferred embodiment, wash steps will occur at 68° C. in 15 mM NaCl, 1.5 mM sodium citrate, and 0.1% SDS. A skilled artisan can readily determine and vary the stringency conditions appropriately to obtain a clear and detectable hybridization signal.

Recombinant DNA Molecules Having Apex-encoding Nulceic Acid Molecules

Also provided are recombinant DNA molecules (rDNAs) that include APEX-encoding sequences as herein described, or fragments thereof. As used herein, a rDNA molecule is a DNA molecule that has been subjected to molecular manipulation in vitro. Methods for generating rDNA molecules are well known in the art, for example, see Sambrook et al., Molecular Cloning (1989). In the preferred rDNA molecules of the present invention, an APEX-encoding DNA sequence that encodes an APEX protein or a fragment of APEX, is operably linked to one or more expression control sequences and/or vector sequences. The rDNA molecule can encode either the entire APEX protein, or can encode a fragment of the APEX protein having APEX activity.

The choice of vector and/or expression control sequences to which the APEX-encoding sequence is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the APEX-encoding sequence included in the rDNA molecule.

Expression control elements that are used for regulating the expression of an operably linked protein encoding sequence are known in the art and include, but are not limited to, inducible promoters, constitutive promoters, secretion signals, enhancers, transcription terminators and other regulatory elements. Preferably, an inducible promoter that is readily controlled, such as being responsive to a nutrient in the host cell's medium, is used.

In one embodiment, the vector comprising an APEX-encoding nucleic acid molecule will include a prokaryotic replicon, i.e., a DNA sequence having the ability to direct autonomous replication and maintenance of the recombinant DNA molecule intrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or viral promoter capable of directing the expression (transcription and translation) of the APEX-encoding sequence in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Various viral vectors well known to those skilled in the art may also be used, such as, for example, a number of well-known retroviral and adenoviral vectors.

Expression vectors compatible with eukaryotic cells, preferably those compatible with vertebrate cells, can also be used to express rDNA molecules that include an APEX-encoding sequence. Eukaryotic cell expression vectors are well known in the art and are available from several commercial sources. Typically, such vectors are provided containing convenient restriction sites for insertion of the desired DNA segment. Typical of such vectors are PSVL and pKSV-10 (Pharmacia, Uppsala, Sweden), pBPV-1/pML2d (International Biotechnologies, Inc.), pTDT1 (ATCC, #31255), and similar eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. Alternatively, the selectable marker can be present on a separate plasmid, and the two vectors are introduced by co-transfection of the host cell, and selected by culturing in the presence of the appropriate drug for the selectable marker.

In accordance with the practice of the invention, the vector can be a plasmid, cosmid or phage vector encoding the cDNA molecule discussed above. Additionally, the invention provides a host-vector system comprising the plasmid, cosmid or phage vector transfected into a suitable eukaryotic host cell. Examples of suitable eukaryotic host cells include a yeast cell, a plant cell, or an animal cell, such as a mammalian cell. The host-vector system is useful for the production of an APEX protein. Alternatively, the host cell can be prokaryotic, such as a bacterial cell.

Recombinant Methods of Generating Apex Proteins

The invention further provides methods for producing an APEX protein using APEX-encoding nucleic acid molecules herein described. In general terms, the production of a recombinant APEX protein typically involves the following steps:

First, a nucleic acid molecule is obtained that encodes an APEX protein or a fragment thereof, such as the nucleic acid molecule depicted in FIGS. 5, 6, or 7. The APEX-encoding nucleic acid molecule is then preferably placed in an operable linkage with suitable expression control sequences, as described above, to generate an expression unit comprising the APEX-encoding sequence. The expression unit is used to transform a suitable host and the transformed host is cultured under conditions that allow the production of the APEX protein. The APEX protein is isolated from the medium or from the cells; recovery and purification of the protein may not be necessary in some instances where some impurities may be tolerated.

Each of the foregoing steps may be done in a variety of ways. For example, the desired coding sequences may be obtained from genomic fragments and used directly in an appropriate host. The construction of expression vectors that are operable in a variety of hosts is accomplished using an appropriate combination of replicons and control sequences. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with APEX-encoding sequences to produce an APEX protein.

In order to express a biologically active APEX, the nucleotide sequence encoding apex or its functional equivalent, is inserted into an appropriate expression vector, i.e., a vector which contains the necessary elements for the transcription and translation of the inserted coding sequence.

Methods which are well known to those skilled in the art can be used to construct expression vectors comprising an apex coding sequence and appropriate transcriptional or translational controls. These methods include in vitro recombinant DNA techniques, synthetic techniques and in vivo recombination or genetic recombination. Such techniques are described in Sambrook et al (1989) in: Molecular Cloning, A Laboratory Manual, Cold Spring Harbor Press, Plainview N.Y. and Ausubel F M et al. (1989) in: Current Protocols in Molecular Biology, John Wiley & Sons, New York, N.Y.

A variety of expression vector/host systems may be utilized to contain and express an apex coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV), or transformed with bacterial expression vectors (e.g., Ti or pBR322 plasmid), or animal cell systems.

Human artificial chromosomes (HACs) may also be employed to deliver larger fragments of DNA than can be obtained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

The “control elements” or “regulatory sequences” of these systems vary in their strength and specificities and are those nontranslated regions of the vector, enhancers, promoters, and 3′ untranslated regions, which interact with host cellular proteins to carry out transcription and translation. Depending on the vector system and host utilized, any number of suitable transcription and translation elements, including constitutive and inducible promoters, may be used. For example, when cloning in bacterial systems, inducible promoters such as the hybrid IacZ promoter of the BLUESCRIPT phagemid (Stratagene, LaJolla, Calif.) or PSPORTI (Gibco BRL) and ptrp-lac hybrids, and the like, may be used. The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from the mammalian genes or from mammalian viruses are appropriate. If it is necessary to generate a cell line that includes multiple copies of apex, vectors based on SV40 or EBV may be used with an appropriate selectable marker.

The invention also provides chimeric or fusion proteins. As used herein, a 'chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the N-terminus or C-terminus of the polypeptide of the invention.

Heterologous protein and peptide moieties may facilitate purification of fusion protein using commercially available affinity matrices. In addition, a chimeric APEX protein containing a heterologous moiety that can be recognized by a commercially available antibody may facilitate the screening of peptide libraries for inhibitors of APEX activity. Such heterologous moieties include, but are not limited to glutathione-S-transferase (GST), immunoglobulin (Ig), maltose binding protein (MBP), thioredoxin (Trx), calmadulin binding protein (CBP), 6-His, FLAG, and hemaglutinin (HA). A fusion protein may be engineered to contain a proteolytic cleavage site located between the APEX encoding sequences and the heterologous protein sequence, so that the APEX sequences may be cleaved away from the heterologous moiety following purification. Methods for fusion protein expression and purification are discussed in Ausubel (1995, supra, ch 10). A variety of commercially available kits may also be used to facilitate expression and purification of fusion protein.

In one embodiment, the fusion protein is a GST-fusion protein in which the polypeptides of the invention are fused to the C-tenninus of GST sequences. In a more preferred embodiment, the fusion protein is an immunoglobulin fusion protein in which all or parts of a polypeptide of the invention is fused to sequence derived from a member of the immunoglobulin protein family. The fusion proteins of the invention can facilitate the purification of recombinant polypeptides of the invention. The immunoglobulin fusion proteins of the invention can also increase the solubility of the polypeptides of the invention. The immunoglobulin fusion protein of the invention can be incorporated into pharmaceutical compositions and administered to a subject to inhibit an interaction between a ligand (soluble or membrane bound) and a protein on the surfaces of cells (receptor), to thereby suppress signal transduction in vivo. Inhibition of ligand-receptor interactions may be useful therapeutically, both for treating proliferative and differentiative disorders and for modulating (e.g. promoting or inhibiting) cell survival. The immunoglobulin fusion proteins of the invention can be used as immunogens to produce antibodies directed against polypetides of the invention in a subject, to purify ligands and in screening assays to identify molecules which inhibit the interaction of receptors with ligands.

In bacterial systems, a number of expression vectors may be selected depending upon the use intended for the APEX proteins. For example, when large quantities of APEX proteins are needed for the induction of antibodies, vectors which direct high level expression of fusion proteins that are readily purified, may be desirable. Such vectors include, but are not limited to, the multifunctional E. coli cloning and expression vectors such as BLUESCRIPT (Stratagene), in which the APEX coding sequence may be ligated into the vector in frame with sequences for the amino-terminal Met and the subsequent 7 residues of β-galactosidase so that a hybrid protein is produced; pIN vectors (Van Heeke & Schuster (1989) J Biol Chem 264:5503-5509); and the like. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads, followed by elution in the presence of free glutathione. Proteins made in such systems are designed to include heparin, thrombin or factor XA protease cleavage sites so that the cloned polypeptide of interest can be released from the GST moiety at will.

In the yeast, Saccharomyces cerevisiae, a number of vectors containing constitutive or inducible promoters such as β-factor, alcohol oxidase and PGH may be used. For reviews, see Ausubel et al (supra) and Grant et al (1987) Methods in Enzymology 153:516-544.

In cases where plant expression vectors are used, the expression of a sequence encoding an APEX protein may be driven by any of a number of promoters. For example, viral promoters such as the 35S and 19S promoters of CaMV (Brisson et al (1984) Nature 310:511-514) may be used alone or in combination with the omega leader sequence from TMV (Takamatsu et al (1987) EMBO J 6:307-311). Alternatively, plant promoters such as the small subunit of RUBISCO (Coruzzi et al (1984) EMBO J 3:1671-1680; Broglie et al (1984) Science 224:838-843); or heat shock promoters (Winter J and Sinibaldi R M (1991) Results Probl Cell Differ 17:85-105) may be used. These constructs can be introduced into plant cells by direct DNA transformation or pathogen-mediated transfection. For reviews of such techniques, see Hobb, S. or Murry, L E in: McGraw Yearbook of Science and Technology (1992) McGraw Hill New York, N.Y., pp 191-196, or Weissbach and Weissbach (1988) Methods for Plant Molecular Biology, Academic Press, New York, N.Y., pp 421-463.

An alternative expression system which could be used to express APEX proteins is an insect system. In one such system, Autographa californica nuclear polyhedrosis virus (AcNPV) is used as a vector to express foreign genes in Spodoptera frugiperda cells or in Trichoplusia larvae. The APEX coding sequence may be cloned into a nonessential region of the virus, such as the polyhedrin gene, and placed under control of the polyhedrin promoter. Successful insertion of APEX will render the polyhedrin gene inactive and produce recombinant virus lacking coat protein. The recombinant viruses are then used to infect S. frugiperda cells or Trichoplusia larvae in which APEX protein is expressed (Smith et al (1983) J Virol 46:584; Engelhard E K et al (1994) Proc Nat Acad Sci 91:3224-7).

In mammalian host cells, a number of viral-based expression systems may be utilized. In cases where an adenovirus is used as an expression vector, an apex coding sequence may be ligated into an adenovirus transcription/translation complex consisting of the late promoter and tripartite leader sequence. Insertion in a nonessential E1 or E3 region of the viral genome will result in a viable virus capable of expressing APEX in infected host cells. (Logan and Shenk (1984) Proc Natl Acad Sci 81:3655-59). In addition, transcription enhancers, such as the rous sarcoma virus (RSV) enhancer, may be used to increase expression in mammalian host cells.

Specific initiation signals may also be required for efficient translation of an apex sequence. These signals include the ATG initiation codon and adjacent sequences. In cases where the apex initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al (1994) Results Probl Cell Differ 20:125-62; Bittner et al (1987) Methods in Enzymol 153:516-544).

For long-term, high-yield production of recombinant proteins, stable expression is preferred. For example, cell lines which stably express APEX may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler, M. et al (1977) Cell 11:223-32) and adenine phosphoribosyltransferase genes (Lowy I et al (1980) Cell 22:817-23) which can be employed in tk-negative or aprt-negative cells, respectively. Also, antimetabolite, antibiotic or herbicide resistance can be used as the basis for selection; for example, dhfr which confers resistance to methotrexate (Wigler, M. et al (1980) Proc Natl Acad Sci 77:3567-70); npt, which confers resistance to the aminoglycosides neomycin and G-418 (Colbere-Garapin, F. et al (1981) J Mol Biol 150:1-14) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc Natl Acad Sci 85:8047-51). Recently, the use of visible markers has gained popularity with such markers as anthocyanins, β-glucuronidase and its substrate, GUS, and luciferase and its substrate, luciferin, being widely used not only to identify transformants, but also to quantify the amount of transient or stable protein expression attributable to a specific vector system (Rhodes, C. A. et al (1995) Methods Mol Biol 55:121-131).

Host cells transformed with nucleotide sequences encoding APEX proteins may be cultured under conditions suitable for the expression and recovery of the APEX protein from cell culture. The APEX protein produced by a transformed cell may be secreted or retained intracellularly depending on the sequences and/or the vector used. As will be understood by those skilled in the art, expression vectors containing polynucleotides which encode APEX proteins may be designed to contain signal sequences which direct secretion of APEX proteins through a prokaryotic or eukaryotic cell membrane.

In addition, a host cell strain may be chosen for its ability to modulate expression of the inserted apex sequences or to process the expressed APEX protein in the desired fashion. Such modifications of the APEX polypeptide include, but are not limited to, acetylation, carboxylation, glyosylation, phosphorylation, lipidation, and acylation. Post-translational processing which cleaves a 'prepro” form of the protein may also be used to specify protein targeting, folding, and/or activity. Different host cells which have specific cellular machinery and characteristic mechanisms for post-translational activities (e. g., COS, CHO, HeLa, MDCK, HEK293, and W138), are available from the American Type Tissue Collection (ATCC, Bethesda, Md.), and may be chosen to ensure the correct modification and processing of the foreign APEX protein.

Assays for Identifying Apex Ligands and Other Binding Agents

Another aspect of the invention relates to assays and methods that can be used to detect and identify APEX ligands and other agents and cellular constituents that bind to APEX. Specifically, APEX ligands and other agents and cellular constituents that bind to APEX can be identified by the ability of the APEX ligand or other agent or constituent to bind to APEX and/or the ability to inhibit/stimulate APEX activity. Assays for APEX activity (e.g., binding) using an APEX protein are suitable for use in high throughput screening methods.

In one embodiment, the assay comprises mixing APEX with a test agent or cellular extract. After mixing under conditions that allow association of APEX with the agent or component of the extract, the mixture is analyzed to determine if the agent/component is bound to APEX. Binding agents/components are identified as being able to bind to APEX. Alternatively or consecutively, APEX activity can be directly assessed as a means for identifying agonists and antagonists of APEX activity.

Alternatively, targets that bind to an APEX protein can be identified using a yeast two-hybrid system (Fields, S. and Song, O. (1989) Nature 340:245-246), or using a binding-capture assay (Harlow, supra). Generally, the yeast two-hybrid system is performed in a yeast host cell carrying a reporter gene, and is based on the modular nature of the GAL transcription factor which has a DNA binding domain and a transcriptional activation domain. The two-hybrid system relies on the physical interaction between a recombinant polypeptide that comprises the DNA binding domain and another recombinant polypeptide that comprises the transcriptional activation domain, to reconstitute the transcriptional activity of the modular transcription factor, thereby causing expression of the reporter gene. Either of the recombinant polypeptides used in the two-hybrid system can be constructed to include the APEX-encoding sequence to screen for cellular binding partners of APEX protwin. The yeast two-hybrid system can be used to screen cDNA expression libraries (G. J. Hannon, et al. (1993) Genes and Dev. 7: 2378-2391), and random aptmer libraries (J. P. Manfredi, et al. (1996) Molec. And Cell. Biol. 16: 4700-4709), or semi-random (M. Yang, et al. (1995) Nucleic Acids Res. 23: 1152-1156) aptmers libraries for APEX ligands.

APEX proteins which may be used in the above assays include, but are not limited to, an isolated APEX protein, a fragment of an APEX protein having APEX activity, a cell that has been altered to express an APEX protein, or a fraction of a cell that has been altered to express an APEX protein. Further, the APEX protein can be the entire APEX protein or a defined fragment of the APEX protein having APEX activity. It will be apparent to one of ordinary skill in the art that so long as the APEX protein can be assayed for agent binding, e.g., by a shift in molecular weight or activity, or the expression of a reporter gene in a two-hybrid system.

The method used to identify whether an agent/cellular component binds to an APEX protein will be based primarily on the nature of the APEX protein used. For example, a gel retardation assay can be used to determine whether an agent binds to APEX or a fragment thereof. Alternatively, immunodetection and biochip (e.g., U.S. Pat. No. 4,777,019) technologies can be adopted for use with the APEX protein. A skilled artisan can readily employ numerous art-known techniques for determining whether a particular agent binds to an APEX protein.

Agents and cellular components can be further tested for the ability to modulate the activity of an APEX protein using a cell-free assay system or a cellular assay system. As the activities of the APEX protein become more defined, functional assays based on the identified activity can be employed.

Agents that bind an APEX protein, such as an APEX antibody, can be used to modulate the activity of APEX, to target anticancer agents to appropriate mammalian cells, or to identify agents that block the interaction with APEX. Cells expressing APEX can be targeted or identified by using an agent that binds to APEX.

How the APEX binding agents will be used depends on the nature of the APEX binding agent. For example, an APEX binding agent can be used to deliver conjugated toxins, such a diphtheria toxin, cholera toxin, ricin or pseudomonas exotoxin, to an APEX expressing cell; modulate APEX activity; to directly kill APEX expressing cells, or in screens to identify competitive binding agents. For example, an APEX inhibitory agent can be used to directly inhibit the growth of APEX expressing cells, whereas an APEX binding agent can be used as a diagnostic agent.

As used herein, an agent is said to antagonize APEX activity when the agent reduces APEX activity. The preferred antagonist will selectively antagonize APEX, not affecting any other cellular proteins. Further, the preferred antagonist will reduce APEX activity by more than 50%, more preferably by more than 90%, most preferably eliminating all APEX activity.

As used herein, an agent is said to agonize APEX activity when the agent increases APEX activity. The preferred agonist will selectively agonize APEX, not affecting any other cellular proteins. Further, the preferred antagonist will increase APEX activity by more than 50%, more preferably by more than 90%, most preferably, more than doubling APEX activity.

Agents that are assayed in the above method can be randomly selected or rationally selected or designed. As used herein, an agent is said to be randomly selected when the agent is chosen randomly without considering the specific sequences of the APEX protein. An example of randomly selected agents is the use of a chemical library or a peptide combinatorial library, or a growth broth of an organism or plant extract.

As used herein, an agent is said to be rationally selected or designed when the agent is chosen on a nonrandom basis that takes into account the sequence of the target site and/or its conformation in connection with the agent's action. Agents can be rationally selected or rationally designed by utilizing the peptide sequences that make up the APEX protein. For example, a rationally selected peptide agent can be a peptide whose amino acid sequence is identical to a fragment of an APEX protein having APEX activity.

The agents can be, as examples, peptides, small molecules, and vitamin derivatives, as well as carbohydrates. A skilled artisan can readily recognize that there is no limit as to the structural nature of the agents used in the present screening method. One class of agents of the present invention is peptide agents whose amino acid sequences are chosen based on the amino acid sequence of the APEX protein. Small peptide agents can serve as competitive inhibitors of APEX protein assembly.

Peptide agents can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies immunoreactive with selected domains or regions of the APEX protein. As described above, antibodies are obtained by immunization of suitable mammalian subjects with peptides, comprising as antigenic regions, those portions of the APEX protein intended to be targeted by the antibodies. Regions of particular interest may include the extracellular domain of an APEX polypeptide. Such agents can be used in competitive binding studies to identify second generation inhibitory agents.

The cellular extracts tested in the methods of the present invention can be, as examples, aqueous extracts of cells or tissues, organic extracts of cells or tissues or partially purified cellular fractions. A skilled artisan can readily recognize that there is no limit as to the source of the cellular extract used in the screening method of the present invention.

Uses of the Invention

There are multiple uses of the invention. For example, the nucleic acid molecules of the invention and their encoded proteins (also referred to herein as APEX proteins or protein of the invention), may be employed as molecular weight markers. The molecular weight of each of the gene sequences and proteins can be determined and once determined can be used to compare against other gene sequences and proteins whose molecular weights are unknown. For example, the nucleotide length of apex-1 is 2704 as described in SEQ ID NO. 1; apex-2 is 1516 as described in SEQ ID NO. 2; apex-3 is 1408 as described in SEQ ID NO. 3.

Detection and Mapping of Related Polynucleotide Sequences

The nucleic acid molecules of the invention can be used to map the location of their corresponding genes and other related naturally occuring genomic sequences. The sequence may be mapped to a particular chromosome or to a specific region of the chromosome, and, thus, locate gene regions associated with disease. There are several approaches for chromosome mapping. For example, chromosome mapping can be accomplished by PCR mapping of somatic cell hybrids (D'Eustachio et al. (1983) Science 220:919-924). Other approaches for chromosome mapping include but are not limited to in situ hybridization (Fan et al. (1990) Proc. Natl. Acad. Sci. USA 87: 6223-27), fluorescence in situ hybridization (FISH) of a DNA sequence to a metaphase chromosomal spread (Verma et al., Human Chromosome: A manual of Basic Techniques, Pergamon press, New York, 1988).

In situ hybridization of chromosomal preparation and physical mapping techniques such as linkage analysis using established chromosomal markers can be used to extend genetic maps. Once a disease or syndrome has been crudely localized by genetic linkage to a particular genomic region, any sequences mapping to that region may represent associated or regulatory genes for further investigation. The nucleotide sequences of the invention may also be used to detect differences in the chromosome location due to translocation, inversion, etc. among normal, carrier or affected individuals.

Functional Assays

The molecules of the invention can be used to assess and elaborate functions of APEX proteins by expressing the sequences encoding APEX at physiologically elevated levels in mammalian cell culture systems. For example the cDNA encoding a particular APEX protein is subcloned into a mammalian expression vector containing a strong promoter such as CMV immediate-early promoter, that drives high level of cDNA expression. The recombinant vector containing the apex sequences is cotransfected along with an additional plasmid containing sequences encoding a marker protein, such as Green Fluorescent Protein, into a human cell line, preferably of endothelial or hematopoietic origin, using either liposome formulation, calcium precipitation, or electroporation. Expression of a marker protein provides a means to distinguish transfected cells from nontransfected cells and is a reliable predictor of cDNA expression from the recombinant vector. Flow cytometry (FCM) is used to identify transfected cells expressing the marker protein, and to evaluate properties, for example, their apoptotic state. FCM detects and qualifies the uptake of fluorescent molecules that diagnose events preceding or coincident with cell death. These events include changes in nuclear DNA contents as measured by DNA staining, changes in cell size and granularity, down-regulation of DNA synthesis as measured by decrease in bromodeoxyuridine uptake, alteration in expression of cell surface and intracellular proteins as measured by reactivity with specific monoclonal antibodies, and alteration in plasma membrane composition as measured by the binding of fluorescein-conjugated Annexin V protein to the cell surface.

Diagnostic Uses of the Invention

There are multiple diagnostic uses of the invention. For example, the invention provides methods for diagnosing in a subject, e.g., an animal or human subject, a disease associated with the presence or absence of the APEX protein. In one embodiment, the method comprises quantitatively determining the amount of APEX protein in the sample (e.g., cell or biological fluid sample) using any one or combination of the antibodies of the invention. Then the amount so determined can be compared with the amount in a sample from a normal subject. The presence of a measurably different amount (i.e., the number of APEX proteins in the test sample exceeds or is reduced from the number of APEX proteins in a normal sample) in the samples, indicating the presence of the disease.

In another embodiment, diagnosis involves quantitatively determining in a sample from the subject the amount of RNA transcripts encoding the APEX protein using the nucleic acid molecules of the invention. The amount so determined can be compared with the amount of RNA in a sample from a normal subject. Once again, the presence of a measurably different amount indicates the presence of a disease associated with the over- or under-abundance of APEX-encoding transcripts.

Additionally, the invention provides methods for monitoring the course of disease or disorders associated with APEX in a subject by measuring the amount of APEX in a sample from the subject at various points in time. This is done for purposes of determining a change in the amount of APEX in the sample e.g., to determine whether the change is a small change in the amount or a large change, i.e., overexpression of APEX. In one embodiment, the method comprises quantitatively determining in a first sample from the subject the presence of an APEX protein and comparing the amount so determined with the amount present in a second sample from the subject, such samples being taken at different points in time, a difference in the amounts determined being indicative of the course of the disease.

In another embodiment, monitoring is effected by quantitatively determining in a first sample from the subject the presence of an apex RNA transcript and comparing the amount so determined with the amount present in a second sample from the subject, such samples being taken at different points in time, a difference in the amounts determined being indicative of the course of the disease associated with apex expression.

As a further embodiment, the diseases or disorders associated with APEX can be monitored in a sample by detecting an increase in or increased APEX gene copy number. An increase in or increased APEX gene copy number is important because it may correlate with poor outcome.

The diagnostic sample can be from an animal or a human. Further, the sample can be a cell sample. For example, using the methods of the invention, spleen, lymph node, thymus, bone marrow, liver, heart, brain, placenta, lung, skeletal muscle, kidney and pancreas can be evaluated for the presence of disease. Alternatively, the sample can be a biological fluid, e.g., urine, blood sera or plasma.

In accordance with the practice of the invention, detection can be effected by immunologic detection means involving histology, blotting, ELISA, and ELIFA. When the sample is a tissue or cell sample it can be formalin-fixed, paraffin-embedded or frozen.

The invention additionally provides methods of determining a difference in the amount and distribution of APEX in tissue sections from a neoplastic tissue to be tested relative to the amount and distribution of APEX in tissue sections from a normal tissue. In one embodiment, the method comprises contacting both the tissue to be tested and the normal tissue with a monoclonal antibody that specifically forms a complex with APEX, and thereby detecting the difference in the amount and distribution of APEX.

Further, the invention provides a method for diagnosing a neoplastic or preneoplastic condition in a subject. This method comprises obtaining from the subject a sample of a tissue, detecting a difference in the amount and distribution of APEX in the using the method above, a distinct measurable difference being indicative of such neoplastic or preneoplastic condition.

In accordance with the practice of the invention, the antibody can be directed to the epitope to which any of the monoclonal antibodies of the invention is directed. Further, the tissue sample can be from, for example, the spleen, lymph node, thymus, bone marrow, liver, heart, brain, placenta, lung, skeletal muscle, kidney, pancreas.

The invention also provides methods of detecting and quantitatively determining the concentration of APEX in a biological fluid sample. In one embodiment the method comprises contacting a solid support with an excess of one or more monoclonal antibodies which forms (preferably, specifically forms) a complex with APEX under conditions permitting the monoclonal antibody to attach to the surface of the solid support. The resulting solid support to which the monoclonal antibody is attached is then contacted with a biological fluid sample so that the APEX in the biological fluid binds to the antibody and forms an APEX-antibody complex. The complex can be labeled directly or indirectly with a detectable marker. Alternatively, either the APEX or the antibody can be labeled before the formation of the complex. The complex can then be detected and quantitatively determined thereby detecting and quantitatively determining the concentration of APEX in the biological fluid sample. A high or low concentration of APEX in the sample relative to normal cells indicates a neoplastic or preneoplastic condition.

In accordance with the practice of the invention, the biological fluid includes, but is not limited to tissue extract, urine, blood, serum, and phlegm. Further, the detectable marker includes but is not limited to an enzyme, biotin, a fluorophore, a chromophore, a heavy metal, a paramagnetic isotope, or a radioisotope.

Further, the invention provides a diagnostic kit comprising an antibody that recognizes and binds APEX (an anti-APEX antibody); and a conjugate of a detectable label and a specific binding partner of the anti-APEX antibody. In accordance with the practice of the invention the label includes, but is not limited to, enzymes, radiolabels, chromophores and fluorescers.

Methods to extend the DNA sequence from an oligonucleotide primer annealed to the DNA template of interest have been developed for both single-stranded and double-stranded templates. Chain termination reaction products are separated using electrophoresis and detected via their incorporated, labeled precursors. Recent improvements in mechanized reaction preparation, sequencing and analysis have permitted expansion in the number of sequences that can be determined per day. Preferably, the process is automated with machines such as the Hamilton Micro Lab 2200 (Hamilton, Reno, Nev.), Peltier Thermal Cycler (PTC200; MJ Research, Watertown Mass.) and the ABI 377 DNA sequencers (Perkin Elmer).

The nucleotide sequence of APEX may be extended utilizing partial nucleotide sequence, and various methods known in the art, to detect upstream sequences such as promoters and regulatory elements. Useful nucleotide sequences may be joined to APEX in an assortment of cloning vectors, e.g., plasmids, cosmids, lambda phage derivatives, phagemids, and the like, that are well known in the art. In general, these vectors will contain an origin of replication functional in at least one organism, convenient restriction endonuclease sensitive sites, and selectable markers for the host cell.

The nucleic acid molecules of the invention and their encoded proteins, may be employed in diagnostic embodiments. For example, the amount of such sequences present within a biological sample, such as blood, serum or a swab from nose, ear or throat, may be determined by means of a molecular biological assay to determine the level of nucleic acid complementary to the gene sequence of the invention, or even by means of an immunoassay to determine the level of one of the products encoded by the gene.

In a molecular biological method for detecting the nucleotide sequences of the invention, one would obtain nucleic acid molecules from a suitable sample and analyze the nucleic acid molecules, using a nucleic acid probe, to identify a specific nucleotide sequence that is complementary to the probe. The complementary nucleic acid molecule will generally be identified by sequence, which method generally includes either; identifying a transcript with a corresponding or complementary sequence, e.g., by Northern or Southern blotting using an appropriate probe or; by identifying a transcript with two or more shorter primers and amplifying with PCR technology.

To conduct such a diagnostic method, one would generally obtain nucleic acid molecules from the sample and contact the nucleic acid molecules with a nucleic acid probe corresponding thereto, under conditions effective to allow hybridization of substantially complementary nucleotide sequences, and then detect the presence of any hybridized substantially complementary nucleic acid complexes that formed.

The presence of a substantially complementary nucleotide sequence in a sample, or a significantly increased level of such a sequence, in comparison to the levels in a normal or “control” sample, will thus be indicative of a sample having the nucleotide sequence of the invention. Here, substantially complementary nucleotide sequences are those that have relatively little sequence divergence and that are capable of hybridizing to the sequences disclosed herein under standard high stringency conditions.

As used herein, the term “increased levels” is used to describe a significant increase in the amount of the gene sequence of the invention detected in a given sample in comparison to that observed in a control sample, e.g., an equivalent sample from a normal healthy subject.

A variety of hybridization techniques and systems are known that can be used in connection with the detection aspects of the invention, including diagnostic assays such as those described in Falkow et al., U.S. Pat. No. 4,358,535.

In general, the “detection” of the nucleotide sequence of the invention is accomplished by attaching or incorporating a detectable label into the nucleic acid molecule used as a probe and “contacting” a sample nucleic acid molecule with the labeled probe. In such processes, an effective amount of the labeled probe is brought into direct juxtaposition with a composition containing the target nucleotide sequence. Hybridized nucleic acid complexes may then be identified by detecting the presence of the label, for example, by detecting a radio, enzymatic, fluorescent, or even chemiluminescent label.

Many suitable variations of hybridization technology are available for use in the detection of target nucleotide sequences, as will be known to those of skill in the art. These include, for example, in situ hybridization, Southern blotting and Northern blotting. In situ hybridization describes the techniques wherein the target nucleotide sequences contacted with the probe sequences are those located within one or more cells, such as cells within a clinical sample or even cells grown in tissue culture. As is well known in the art, the cells are prepared for hybridization by fixation, e.g. chemical fixation, and placed in conditions that allow for the hybridization of a detectable probe with nucleotide sequences located within the fixed cell.

Alternatively, target nucleic acid molecules may be separated from a cell or sample prior to contact with a probe. Any of the wide variety of methods for isolating target nucleic acid molecules may be employed, such as cesium chloride gradient centrifugation, chromatography (e.g., ion, affinity, magnetic), phenol extraction and the like. Most often, the isolated nucleic acid molecules will be separated, e.g., by size, using electrophoretic separation, followed by immobilization onto a solid matrix, prior to contact with the labeled probe. These prior separation techniques are frequently employed in the art and are generally encompassed by the terms “Southern blotting” and “Northern blotting”.

It is possible to detect the nucleic acid molecules of the invention using a method based upon PCR technology. To conduct such a diagnostic method, one would generally obtain sample nucleic acid molecules from a suitable source and contact the sample nucleic acid molecules with two probes or primers corresponding to the apex nucleotide sequence disclosed herein, under conditions which allow for hybridization and polymerization to occur. A pair of probes, one corresponding to the 5′ flanking region and the other corresponding to the 3′ flanking region, would be sufficient to detect the nucleic acid molecules of the invention in a sample and may even be used to quantitate the amount present.

The invention also encompasses diagnostic kits for carrying out the methods disclosed above. In one embodiment, the diagnostic kit comprises (a) an APEX monoclonal antibody and (b) a conjugate of a specific binding partner for APEX antibody and a label for detecting bound antibody. In another embodiment, the diagnostic kit comprises a conjugate of a monoclonal antibody to the invention and a label capable of producing a detectable signal. The reagents may also include ancillary agents such as buffering agents and protein stabilizing agents, e.g. polysaccharides and the like. The diagnostic kit may further comprise, where necessary, other components of the signal-producing system including agents for reducing background interference, control reagents, an apparatus for conducting a test, etc. In another embodiment, the diagnostic kit comprises polynucleotide probes and a label capable of producing a detectable signal. Ancillary agents as mentioned above may also be present.

Therapeutic Uses of the Invention

Structural similarity in the context of sequences and motifs between APEX and proteins defined by CD antigens suggests that APEX proteins may be a potential target for diseases such as inflammation, cancer, and immune disorders. Therapeutic uses of APEX proteins include inhibition of leukocyte function in autoimmune diseases, inhibition of rejection of solid organ and bone marrow transplants (BMT), and inhibition of graft versus host disease following BMT. In the event that an anti-APEX antibody, such as anti-Apex-1 or anti-Apex-2 stimulates the immune system, therapeutic uses include but are not limited to immune system enhancement for vaccine development, oncological uses (anti-cancer immunotherapy), and immune deficiency syndromes including HIV.

In the treatment of cancer and immune disorders associated with decreased expression or activity of APEX, it is desirable to provide the protein, or to increase the expression or activity of APEX. In the treatment of above conditions associated with increased expression or activity of APEX, it is desirable to decrease the expression or activity of APEX. In certain diseases, APEX expression may remain constant, but using an agonist/antagonist may be therapeutically useful to modulate immune cell signaling.

The present invention provides a method for treating or preventing a disorder associated with decreased expression or activity of APEX in an individual. Examples of such disorders include, but are not limited to, immune disorders such as arteriosclerosis, asthma, arteriosclerosis, autoimmune anemia, acquired immunodeficiency syndrome (AIDS), bursitis, cholecystitis, cirrhosis, Crohn's disease; atopic dermatitis, diabetes mellitus, emphysema, atrophic gastritis, inflammatory bowel disease, multiple sclerosis, myasthenia gravis, myocardial or pericardial inflammation, osteoarthritis, osteoporosis, psoriasis, Reiter's syndrome, rheumatoid arthritis, systemic lupus erythematosus; and cancers including adenocarcinoma, leukemia, lymphoma, melanoma, myeloma, sarcoma, teratocarcinoma, cancers of adrenal gland, bladder, bone, bone marrow, breast, cervix, gall bladder, gastointestinal tract, kidney, liver, lung, muscle, ovary, pancreas, prostrate, salivary glands, skin, spleen, testis, thymus, thyroid, and uterus. The method comprises administering to such an individual a composition comprising a therapeutically effective amount of APEX or an agonist thereof. For example, a condition caused by a decrease in the standard or normal expression level of APEX in an individual, can be treated by administering to such an individual a pharmaceutical composition, comprising an amount of APEX polypeptide or an agonist, so as to increase the activity level of APEX in such an individual.

The present invention further provides a method for treating or preventing a disorder associated with increased expression or activity of APEX in an individual. Examples of such disorders include, but are not limited to those described above. The method of the invention comprises administering to such an individual a composition comprising a therapeutically effective amount of APEX antagonist. The preferred antagonists for use in the present invention are APEX-specific antibodies, antisense, or ribozyme molecules. The antisense molecules may be designed to block translation of mRNA by preventing the transcript from binding to ribosomes. Antisense oligonucleotides, specifically those that are derived from the transcription initiation site, e.g., between about −10 and +10 from the start site, are preferred to inhibit production of APEX.

In addition, ribozymes may also be used to catalyze the specific cleavage of RNA. The mechanism of ribozyme action involves sequence-specific hybridization of the ribozyme molecule to complementary target RNA, followed by endonuclease cleavage. For example, engineered hammerhead motif ribozyme molecules (Haselhoff and Gerlach (1988) Nature 334:585-591) may specifically and efficiently catalyze endonucleolytic cleavage of sequences encoding APEX.

In certain diseases, APEX expression may remain constant, but using an agonist/antagonist may be therapeutically useful to modulate immune cell signaling. For example, if APEX is a “negative” signaling molecule, an agonist is immunosuppressive. On the other hand, if APEX turns cells on, an antagonist is immunosuppressive. Thus, APEX proteins may not be specifically involved in the disease, but manipulation of APEX signaling may be therapeutically beneficial.

The polynucleotides of the invention may also be used as reagents for treating a patient using the gene therapy approach. One method for ex vivo gene therapy may employ transplanting onto a patient, fibroblasts which are capable of expressing APEX polypeptides. Generally, fibroblasts are obtained from a subject by skin biopsy and infected with producer cells that produce infectious virus particles containing the apex gene of the invention. The producer cells for this method are prepared by transduction of packaging cells with a retroviral vector, e.g., Maloney murine sarcoma virus (MSV) containing the apex sequences. If the titer of the virus is high, then virtually all fibroblasts will be infected and no selection is needed. However, if the viral titer is low, then it is necessary to use a retroviral vector that has a selectable marker, such as neomycin. Once the fibroblasts have been efficiently infected, the fibroblasts are analyzed to determine if the APEX protein is produced. The engineered fibroblasts are then transplanted onto the host, either alone or after having been grown to confluence on microcarrier beads.

Another aspect of present invention is using in vivo gene therapy methods to treat APEX-associated disorders, diseases, and conditions. The in vivo gene therapy relates to introduction of naked nucleic acid (DNA, RNA, or antisense DNA or RNA) corresponding to apex sequences into a host to increase or decrease the expression of APEX polypeptides. The apex polynucleotides may be operatively linked to a promoter or any other genetic elements that may be necessary for the expression of APEX proteins by the target tissue. Such gene therapy methods are known in the art (Tabata et al., Cardiovasc. Res. (1997) 35:470-479, Wolff et al. (1997) Neuromuscul. Disord. 7:314-318, Schwarz et al. (1996) Gene Ther. 3:405-411). For a detailed description of the delivery techniques and gene therapy methods see U.S. Pat. Nos. 5,693,622; 5,705,151; and 5,580,859.

Endogenous apex gene expression can also be reduced by inactivating or “knocking out” the desired apex gene and/or its promoter using targeted homologous recombination. Methods for generating knock-out animals that fail to express a functional protein molecule are well known in the art (Capechi, Science (1989) 244:1288-1292, Thompson et al., Cell (1989) 5:313-321). Knock-out animals of the invention have uses which include, but are not limited to, animal model system useful for studying in vivo functions of APEX molecules, studying conditions and/or disorders associated with aberrant APEX expression, and in screening for compounds effective in ameliorating such conditions and/or disorders.

Pharmaceutical Compositions of the Invention

The invention includes pharmaceutical compositions for use in the treatment of APEX-associated diseases comprising a pharmaceutically effective amount of an APEX protein and a pharmaceutically acceptable carrier.

In one embodiment, the pharmaceutical compositions may comprise an APEX antibody, either unmodified, conjugated to a therapeutic agent (e.g., drug, toxin, enzyme or second antibody) or in a recombinant form (e.g., chimeric or bispecific). The compositions may additionally include other antibodies or conjugates (e.g., an antibody cocktail).

The antibody compositions of the invention can be administered using conventional modes of administration including, but not limited to, intravenous, intraperitoneal, oral, intralymphatic or administration directly into the tumor. Intravenous administration is preferred.

The compositions of the invention may be in a variety of dosage forms which include, but are not limited to, liquid solutions or suspensions, tablets, pills, powders, suppositories, polymeric microcapsules or microvesicles, liposomes, and injectable or infusible solutions. The preferred form depends upon the mode of administration and the therapeutic application.

The compositions also preferably include conventional pharmaceutically acceptable carriers and adjuvants known in the art such as human serum albumin, ion exchangers, alumina, lecithin, buffer substances such as phosphates, glycine, sorbic acid, potassium sorbate, and salts or electrolytes such as protamine sulfate.

The most effective mode of administration and dosage regimen for the compositions of this invention depends upon the severity and course of the disease, the patient's health and response to treatment and the judgment of the treating physician. Accordingly, the dosages of the compositions should be titrated to the individual patient.

The following examples are presented to illustrate the present invention and to assist one of ordinary skill in making and using the same. It will be clear that the invention may be practiced otherwise than as particularly described in the foregoing description and examples. It should be understood that these examples are for illustrative purposes only and are not intended in any way to otherwise limit the scope of the invention.

### EXAMPLE 1

Isolation and Characterization of Apex-1

Production of GM-CSf/IL-4 Differentiated Monocyte and THP1 Cells

Human monocytes were obtained from peripheral blood mononuclear cells by elutriation. The isolated monocytes were resuspended in RPMI 1640 medium containing 10% fetal bovine serum (Hyclone, Logan, Utah) supplemented with penicillin/streptomycin, 2 mM glutamine, 1% non-essential amino acids, 1 mM sodium pyruvate IL-4 (75 ng/ml) and GM-CSF (15 ng/ml) (each from Gibco BRL, Grand Island, N.Y.) at a cell concentration of 5×10⁵/ml. Cells were incubated in tissue culture flasks at 37° C., 5% CO₂ for seven days. Following the incubation period, the non-adherent cells were removed from the flask.

THP1 human monocytes were grown to a final concentration of 5×10⁵ cells/ml in RPMI 1640 medium containing 10% fetal bovine serum supplemented with penicillin/streptomycin and 2 mM glutamine.

GM-CSF/IL-4 differentiated human peripheral blood mononuclear cells (2×10⁸) and THP1 human monocytes (2×10⁸) were washed twice with PBS (Gibco BRL) at 4° C.

RNA Isolation

2×10⁸ GM-CSF/IL-4 differentiated human peripheral blood mononuclear cells and 2×10⁸ THP1 human monocytes were washed twice with PBS (Gibco BRL, Grand Island, N.Y.) at 4°Celsius. Poly A+RNA was isolated directly using Fast Track 2.0™ (Invitrogen, Carlsbad, Calif.).

Construction of the Subtraction Library

The PCR-select cDNA subtraction kit™ (Clontech, Palo Alto, Calif.) was used to generate a subtraction library from GM-CSF/IL-4 human monocyte poly A+ RNA (tester) and THP1 human monocyte poly A+ RNA (driver). Ten secondary PCR reactions were combined and run on a 2% agarose gel. Fragments ranging from approximately 0.3 kb -1.5 kb were gel purified using the QIAgen gel extraction kit (QIAgen Inc., Valencia, Calif.) and inserted into the TA cloning vector, pCR2.1 (Invitrogen). TOP10F′ competent E. coli (Invitrogen) were transformed and plated on LB plates containing 50 micrograms/ml ampicillin. Clones were isolated and grown in LB broth containing similar concentrations of ampicillin. Plasmid DNAs were sequenced using standard techniques.

A 367 bp clone was isolated and found to have homology to the 5′ region of several members of the CD2 subgroup of the Ig superfamily. A clone which included the remaining sequence of apex-1 was isolated by 3′ PCR amplification using gene specific primers, such as JNF primers (see below). The selection of the sequences of the JNF primers was based on a consensus sequence of an aligned contig sequence that was generated by overlapping the sequences of six public ESTs (EST database) (AA381714, H73135, AA554342, H74227, AA921765, AA765813).

3′-Rapid Amplification of cDNA Ends

In an effort to isolate clones that included the remaining 3′untranslated region (3′-UTR) of apex-1 , rapid amplification of the 3′ ends of apex-1 cDNA was performed using 3′-RACE PCR technology.

First strand cDNA was synthesized from GM-CSF/IL-4 differentiated mRNA using a hybrid oligo dT primer (Zhang, Y. and Frohman, M. A. 1997 Methods Mol. Biol. 69:61-87), termed JNF3 (5′-cca gtg agc aga gtg acg agg act cga gct caa gct ttt ttt ttt ttt ttt t-3′) (SEQ ID NO.: 9) and the Superscript pre-amplification system (Gibco BRL, Grand Island, N.Y.). To amplify apex-1 3′-end sequences, gene-specific primers JNF1 and JNF2 were paired with JNF4 and JNF5, respectively. First round amplification using JNF1 (5′-cag agt acg aca caa tcc c-3′) (SEQ ID NO.: 7) and JNF4 (5′-gag gac tcg agc tca agc-3′) (SEQ ID NO.: 10) was done using 25 cycles at 94 degrees C., 30 sec; 57 degrees C., 30 sec; and 72 degrees C., 1 min. Primary reactions were diluted 50 fold and used in secondary amplification with JNF2 (5′-act cca ctg tgg aaa tac cg-3′) (SEQ ID NO.: 8) and JNF5 (5′-cca gtg agc aga gtg acg-3′) (SEQ ID NO.: 11). JNF1/JNF4 and JNF2/JNF5 amplimers were gel-purified and cloned into pCR2.1 (Invitrogen). The JNF2/JNF5 primer-pair generated an amplimer product that is 1753 bp and spans the remaining translated and 3′-untranslated regions of apex-1.

A full-length contig sequence of apex-1 was generated by aligning the 1753 bp sequence with the 1336 bp contig sequence. A 2704 bp clone which includes the full-length sequence of apex-1 cDNA was isolated by PCR amplification of GM-CSF/IL-4 cDNA using the primer pair Llewellyn4 (5′-ccc aag ctt cca gag agc aat atg gct ggt cc-3′) (SEQ ID NO.:21) and JNF4 (SEQ ID NO.: 10). The full-length fragment was cloned into pCR2.1 and sequenced.

One clone was found to have significant identity with several members of the Ig superfamily CD2 subgroup including CD84 (38%), Ly9 (37%), SLAM (31%).

Transcript Expression of Apex-1 in Various Cell Lines

Several cell lines and human leukocyte populations were analyzed for expression of apex-1 transcripts using RT-PCR analysis. Total RNA was isolated from various B and T cell lines and freshly purified neutrophils. Specifically, total RNA was isolated from brain, heart, lung, liver, kidney, pancreas, small intestine, ovary, testis, prostate, placenta, skeletal muscle, spleen, thymus, bone marrow, tonsil, lymph node, leukocyte, and fetal liver. Additionally, total RNA was isolated from resting T cells, a B cell line (TJ), THP1 monocytes, monocytes stimulated with GM-CSF and IL-4, unactivated NK cells, and unactivated PMN cells.

First-strand cDNA was synthesized using the Superscript Preamplification System (Gibco BRL, Grand Island, N.Y.), as described in the section above. As controls, expression of apex-1 transcripts was tested in GM-CSF/IL-4 differentiated human monocytes (e.g., detectable levels) and THP1 (e.g., no detectable levels). The primers JNF6 (5′-atc ctt tgg cag ctc aca gg-3′) (SEQ ID NO.:12) and JNF7 (5′-ctt cac aga gct tcc tgg c-3′) (SEQ ID NO.: 13) were used to amplify a 613 bp fragment from cDNA that were generated using Superscript Preamplification Kit™ (Gibco BRL, Grand Island, N.Y.).

Northern Analysis

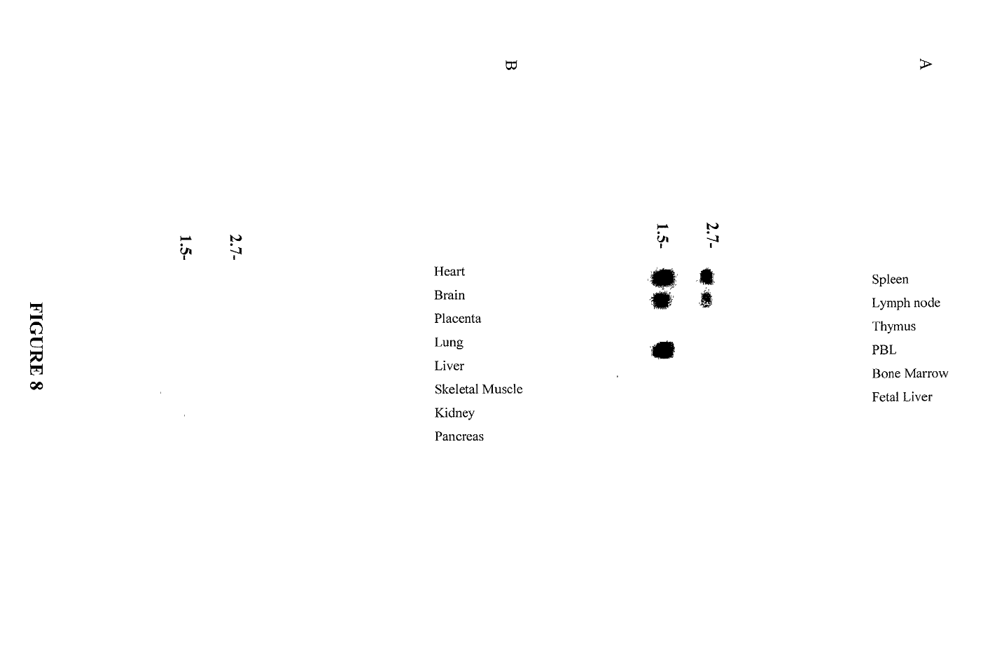

The presence of apex-1 transcripts in various tissues was analyzed by Northern analysis using multiple tissue northern membranes (Human MTNI #7760-1; Human Immune System MTNII #7768-1) purchased from Clontech Laboratories (Palo Alto, Calif.). The apex-1 transcripts were detected by hybridization with [³²P]dCTP-radiolabeled (NEN, Boston, Mass.) random-primed 368 bp apex-1 cDNA probe. The membranes were washed under high stringency conditions (0.1×SSC/0.1% SDS at 65° C.) and exposed for 48 hours at −80 ° C. The results of the Northern blot is shown in FIG. 8.

Results

A full-length cDNA clone which includes the human apex-1 sequence was isolated by PCR amplification of transcripts from a subtraction library that was constructed from GM-CSF/IL-4 differentiated human monocytes minus THP1 human monocyte cell line.

The nucleotide sequence of apex-1 (FIG. 2) includes an open reading frame (ORF) that is predicted to encode a protein 335 amino acid in length that exhibits structural properties shared by members of the CD2 subfamily (Hahn, W. C., et al., 1993 Chapter 5, in: Lymphocyte Adhesion Molecules, ed. Shimizu, Y.). This nucleotide sequence is predicted to encode the APEX-1 protein that includes an N-terminal 22 amino acid hydrophobic signal peptide, a 203 amino acid extracellular domain, a 24 amino acid residue transmembrane domain, and an 86 amino acid intracellular domain (see FIG. 5). The extracellular domain has two Ig-like regions (V-tC2). The V region lacks the typical disulfide bonds, while the C2 region includes two conserved disulfide bridges. There are six potential N-glycosylation sites in the extracellular domain of the sequence N-X-S/T. The intracellular domain includes four tyrosines. Two of the four tyrosines are predicted to form two different SH2 domain binding motifs (Songyang, Z., et al., (1993) Cell 72:767-778; Songyang, Z., et al., (1994) Molec. Cell. Biol. 14:2777-2785). Diversity in the two other tyrosine signaling motifs indicates that APEX-1 may bind a diverse range of kinases.

A data base search for apex-1 related nucleotide sequences demonstrated significant sequence homologies with AJ271869, Z51572 (PCT WO2000011150-A1), Z49571 and Z49572 (PCT WO9967387-A2), Z65041 (PCT 9963088-A2), X41503 (PCT WO9906553-A2), and X00615 (PCT WO9842738-A1). APEX-1 shares structural similarities with members of the CD2 family. Thus, it is postulated that APEX-1 is a cell-surface receptor that regulates adhesion and generates co-stimulatory signals to mediate leukocyte proliferation, differentiation, migration, or activation. It is possible that APEX-1 enhances antigen-specific proliferation and cytokine production, similar to SLAM which is another member of the CD2 subfamily. Thus, APEX-1 may prove to be a potential target for diseases with an inflammatory and autoimmune component indicating that the apex-1 gene encodes a protein that belongs to the Ig superfamily and may be involved in ligand binding and transmitting signals from the cell surface.

The RNA transcript pattern of apex-1 was analyzed by Northern blot of immune and non-immune tissues and revealed the expression of two apex-1 transcripts, measuring 2.7 kb and 1.5 kb in length (FIG. 8). Relatively high levels of the 2.7 kb transcript was detectable in immune tissue, such as spleen and lymph node (FIG. 8A). Lower levels were detectable in peripheral blood lymphocytes (PBL), and very low levels were detectable in bone marrow. In contrast, the 1.5 kb transcript was detectable in both immune and non-immune tissues. Relatively high levels of the 1.5 kb transcript was detectable in spleen, lymph node and PBL. Significantly lower levels of the 1.5 kb transcript was detectable in heart, lung, and placenta (FIG. 8B).

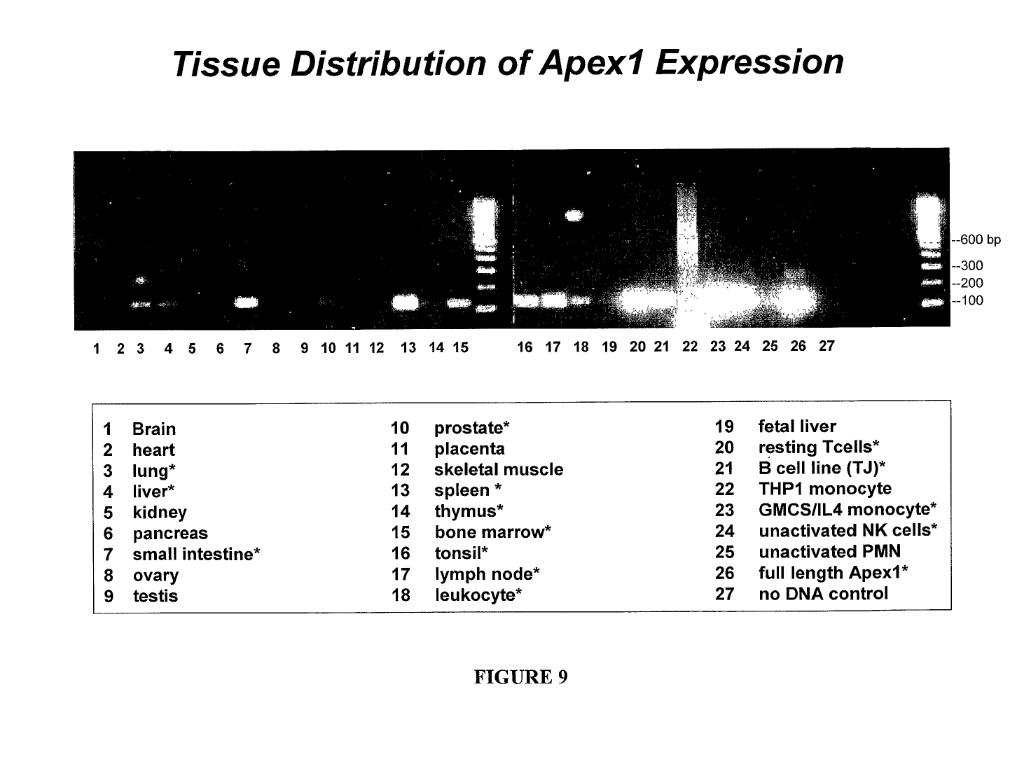

The transcript expression of apex-1 was analyzed in several cell lines and human leukocyte populations by RT-PCR analysis using gene-specific primers that hybridize to the region of apex-1 that encodes the extracellular domain. Transcripts of apex-1 were detectable in cells from tonsil, lymph nodes, whole leukocytes, resting T cells and NK cells, but were not expressed in fresh neutrophils or the THP-1 cell line. Monocytes stimulated with GM-CSF and IL-4 as well as a B cell line (e.g., TJ is from a normal human subject) expressed apex-1 transcripts (FIG. 9).

These results suggest that apex-1 is a new member of the Ig superfamily, which may be involved in transducing signals to APEX-1 -expressing cells.

### EXAMPLE 2

Isolation and Characterization of Apex-2

Production of HSP70-treated Cells

The 70Z/3 murine pre B-cells were grown to a density of 5×10⁵ cells/ml and treated with 1 ug/ml HSP70 (200 ug total) for 1 hour. The cells were harvested and washed. Similar numbers of untreated cells were harvested.

RNA Isolation

Poly A+ RNA was isolated directly from the HSP70-treated and non-treated 70Z/3 cells using the Fast Track 2.0 kit™ (Invitrogen, Carlsbad, Calif.).

Construction of the Subtraction Library

The PCR-select cDNA subtraction kit™ (Clontech, Palo Alto, Calif.) was used to generate a subtraction library from untreated 70Z/3 poly A+ RNA (tester) and HSP70-treated 70Z/3 poly A+ RNA (driver). Secondary PCR reactions were combined and run on a 2% agarose gel. Fragments ranging from approximately 0.3 kb-1.5 kb were gel purified using the QIAgen gel extraction kit (QIAgen Inc., Valencia, Calif.) and inserted into the TA cloning vector, pCR2.1 (Invitrogen). TOP10F′ competent E. coli (Invitrogen) were transformed and plated on LB plates containing 50 micrograms/ml ampicillin. Plasmids were isolated using QIAgen miniprep spin (QIAgen) and sequenced using ABI cycle sequencers (ABI Prism, PE Applied Biosystems).

A 958 bp cDNA clone was isolated which included the sequence of the 3′ region of apex-2. The remainder of the apex-2 sequence was isolated by 5′-RACE PCR using the primer that was supplied by the kit and two gene-specific primers (e.g., JNF).

5′-Rapid Amplification of Apex-2

A DNA fragment, which included the sequence of the 5′ region of apex-2, was isolated using HSP70-treated murine 70Z/3 cDNA and the MARATHON RACE amplification kit from Clontech Laboratories (Palo Alto, Calif.). The gene-specific primer JNF22 (5′-tta acc ttc agg gta atg gg-3′) (SEQ ID NO.:25) and JNF23 (5′-gaa caa tgc aaa tgg cag cg-3′) (SEQ ID NO.:26), and the AP1 primer provided by the MARATHON RACE kit were used to amplify fragments that included sequences in the 5′ region of apex-2. The 5′ amplimer fragment, resulting from the AP1/JNF22 primer pair was ligated to the 958 bp fragment thereby generating a full length cDNA clone of apex-2. A sequence analysis showed that apex-2 is homologous to members of the CD2 subgroup of the Ig superfamily, such as CD84, Ly9, 2B4.

Results

A full length cDNA clone which includes the murine apex-2 sequence was isolated by PCR amplification of transcripts from a murine subtraction library that was constructed from untreated murine 70Z/3 poly A+ RNA minus murine HSP70-treated 70Z/3 poly A+ RNA.

The nucleotide sequence of apex-2 (FIG. 3) includes an open-reading frame that is predicted to encode a protein 351 amino acids in length that exhibits structural properties shared by members of the CD2 subfamily. The predicted APEX-2 protein includes a putative signal peptide of 29 amino acids, a 210 amino acid extracellular domain or region, a 23 amino acid internal hydrophobic segment that represents a transmembrane domain, and an 89 amino acid intracellular (e.g., cytoplasmic) domain (FIG. 6). The extracellular portion is predicted to have two Ig-like domains (a V-set and a C2-set). The predicted extracellular domain has nine Asn-linked glycosylation sites. The predicted cytoplasmic domain has 3 potential SH2 domain binding motifs (YAQV, YSIV, YNQP). The first two motifs may be involved in binding to SHP-2. A database search for apex-2 related sequences demonstrated significant sequence homologies of apex-2 with AF248634 and AF248636 (EMBL/GenBank/DDBJ, submitted Mar. 21, 2000)

APEX-2 shares structural similarities with members of the CD2 family. Thus, like APEX-1, it is postulated that APEX-2 is a cell-surface receptor that regulates adhesion and generates co-stimulatory signals to mediate leukocyte proliferation, differentiation, migration, or activation. It is possible that APEX-2 enhances antigen-specific proliferation and cytokine production, similar to SLAM which is another member of the CD2 subfamily. Thus, APEX-2 may prove to be a potential target for diseases with an inflammatory and autoimmune component indicating that the apex-2 gene encodes a protein that belongs to the Ig superfamily and may be involved in ligand binding and transmitting signals from the cell surface.

### EXAMPLE 3

Obtaining and Characterizing Apex-3

The predicted amino acid sequence of murine APEX-2 was used to search propietary (e.g., Incyte Pharmaceutical) and public (EST database) databases. Two clusters of EST sequences were identified as having considerable homology to murine APEX-2. The first cluster contained 20 overlapping clones from the Incyte database, while the second contained 14 overlapping clones from the EST database. The contig encodes an open reading frame that is homologous to APEX-1, APEX-2, CD84, SLAM and CD48.

Samples of two EST clones, each containing full-length apex-3 sequences, were obtained from a proprietary database and the clones were sequenced. The nucleotide sequence of apex-3 (FIG. 4) includes an open reading frame having homology to apex-2, apex-1 , CD84, SLAM and CD48. The nucleotide sequence of apex-3 is predicted to encode a type-1 trans-membrane protein. APEX-3 includes two Ig-like domains (V-set and C2-set). These are similar to those described previously for APEX-1 and APEX-2. APEX-3 includes: a 22 amino acid signal sequence beginning with the first methionine; an internal hydrophobic segment of 23 amino acids characterizes the putative transmembrane domain and splits the mature protein into two segments; the putative extracellular domain consists of 209 amino acids; and includes 3 potential Asn-glycosylation sites (FIG. 7). The putative cytoplasmic domain is much shorter (31 amino acids) than that seen with other CD2 subgroup members and does not include any SH2-domain binding motifs.

A database search of apex-3 related sequences identified two sequences, Z238457 (PCT WO9940184-A1) and A09042 (PCT WO20001880-A1) that demonstrated significant homology with apex-3.

APEX-3 shares structural similarities with members of the CD2 family. Thus, like APEX-1, it is postulated that APEX-3 is a cell-surface receptor that regulates adhesion and generates co-stimulatory signals to mediate leukocyte proliferation, differentiation, migration, or activation. It is possible that APEX-3 enhances antigen-specific proliferation and cytokine production, similar to SLAM which is another member of the CD2 subfamily. Thus, APEX-3 may prove to be a potential target for diseases with an inflammatory and autoimmune component indicating that the apex-3 gene encodes a protein that belongs to the Ig superfamily and may be involved in ligand binding and transmitting signals from the cell surface.

### EXAMPLE 4

Expression of APEX-1Ig and APEX-2mIg Fusion Proteins

Construction of Apex-1Ig Expression Plasmid

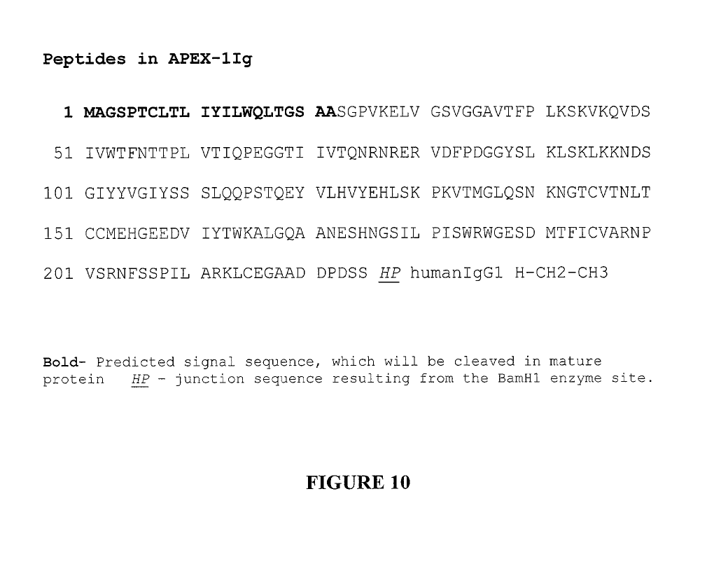

A fusion protein including the putative extracellular domains of APEX-1 and human IgG1, (Apex-1Ig) was produced. cDNA fragments corresponding to the Apex domains were obtained by PCR (forward primer HIIIDS4fp CCC AAG CTT CCA GAG AGC AAT ATG GCT GGT TCC (SEQ ID NO.:35); reverse primer DS4Bamrp C GCG GAT CCG AGG AAT CTG GGT CAT CAG CAG CAC C (SEQ ID NO.:36), PCR product cut with HindIII and BamHI restriction endonucleases), and joined to a cDNA fragment encoding the hinge (H), CH₂ and CH₃ domains of human IgG1 in the vector pCDM7⁻ which was likewise digested with HindIII and BamHI restriction endonucleases) (Starling, G. C et. al. (1996) Eur. J. Immunol.26:738-46). Ligated products were used to transform MC1061/P3 E. coli, which were grown on agar containing 50 ug/ml ampicillin and 15 ug/ml tetracyclin and colonies screened for the appropriate plasmids. Cloned cDNAs were obtained from the bacteria by standard SDS lysis and purification on QIAGEN miniprep columns. The integrity APEX-1-encoding portion of apex-1Ig cDNA clone was confirmed by sequencing. The resulting amino acid sequence of the expressed protein contributed to by the apex-1 cDNA is shown (FIG. 10). The amino terminal secretory sequence was derived from the endogenous apex-1 gene, and the amino acids 1-225 of APEX-1 are present (FIG. 2). The BamHI enzyme digestion site adds His₂₂₆-Pro₂₂₇ amino acids at the junction of the APEX-1 sequence and the H—CH₂—CH₃ Ig portion of the molecule. This construct contained a Thrombin cleavage site which was located immediately C-terminal to the His-Pro residues (Hollenbaugh et al., J. Immunol. Methods (1995) 188: 1-7) to enable separation of APEX-1 extracellular domain from human IgG1 sequence.

Construction of Apex-2mIg Expression Plasmid

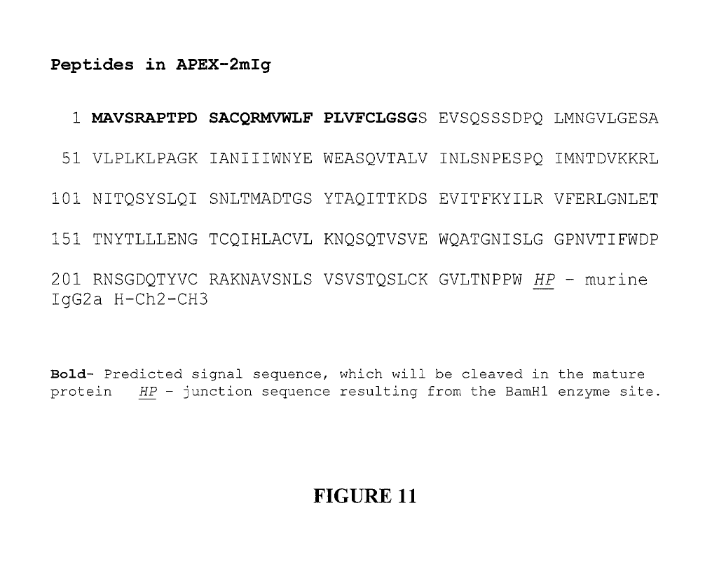

The Apex-2mIg expression plasmid including the entire extracellular domain of Apex-2 obtained by PCR (forward primer Apex2Kpn1FP CCG GGT ACC AAC AGA AAG TCT CAG CGA CAA (SEQ ID NO.:37), reverse primer Apex2BamRP CGC GGA TCC CAG GGT GGA TTA GTT AGA AC (SEQ ID NO.:38), PCR product cut with Kpn1 and BamHI restriction endonucleases), and joined to a cDNA fragment encoding the hinge (H), CH2 and CH3 domains of murine IgG2a in the vector pCDM7⁻ which was likewise digested with Kpn1 and BamHI restriction endonucleases. Ligated products were used to transform MC1061/P3 E. coli, which were grown on agar containing 50 ug/ml ampicillin and 15 ug/ml tetracyclin. Cloned cDNAs were obtained from the bacteria by standard SDS lysis and purification on QIAGEN miniprep columns. The integrity of APEX-2—encoding portion of the apex-2mIg cDNA clone was confirmed by sequencing. The resulting amino acid sequence of the expressed protein contributed to by the Apex-2 cDNA is shown (FIG. 11). The amino terminal secretory sequence was derived from the endogenous apex-2 gene, and the amino acids 1-238 of APEX-2 are present (FIG. 3). The BamHI endouclease restriction site adds His-Pro amino acids at the junction of the APEX-2 and the H—CH₂—CH₃ Ig portion of the molecule.

Expression of APEX-1Ig and APEX-2mIg in COS Cells

The fusion proteins, APEX-1Ig, including the putative extracellular domains of APEX-1 and human Ig, and APEX-2mIg, consisting of the putative extracellular domain of APEX-2 and mouse Ig were produced by transient expression of COS cells in cell factories (Nunc, Roskilde, Denmark) in serum free-DMEM supplemented with 2 mM glutamine. COS cells were grown to approximately 75% confluency in cell factories in DMEM containing 10% fetal bovine serum (FBS). Transient expression of Apex-1Ig and Apex-2mIg was obtained by transfection using the DEAE-Dextran/Chloroquine and purified fusion-protein-encoding vector. DMEM containing 5% Nu-Serum (Becton-Dickinson, Franklin Lakes, N.J.), 1 ug/ml DNA, and DEAE-Dextran/Chloroquine at a final concentration of 400 ug/ml DEAE-Dextran and 100 uM Chloroquine phosphate was added to cells for 4 h at 37 C. in a total volume of 625 ml. Cells were DMSO shocked (1000 ml of 10% DMSO in PBS) for 2 min, prior to overnight culture in 1000 ml of 10% FBS/DMEM. DMEM (1000 ml/factory) supplemented with 2 mM glutamine was substituted at day 1. Cells were cultured in serum-free DMEM for 3 days, and cell-free supernatants were harvested.

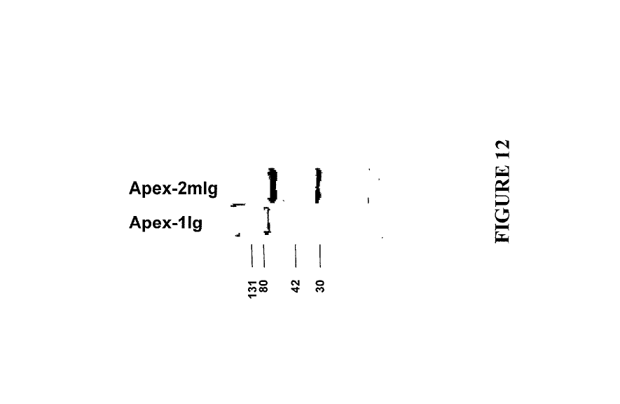

The recombinant APEX-1lg or APEX-2mIg fusion proteins were purified by adsorption to and elution from Protein A Sepharose. Briefly, after removal of cellular debris by low speed centrifugation, medium was applied to a column (approximately 1000 ml of medium/2 ml packed bed volume) of immobilized protein A (Repligen, Cambridge, Mass.) equilibrated with phosphate buffered saline (PBS). After application of the medium, the column was washed with PBS and subsequently binding buffer compatible with the elution buffer (Pierce, Rockford, Ill.), and bound protein was eluted with 15 ml of ImmunoPure Gentle Ag/Ab Elution Buffer (Pierce). Eluted material was both dialyzed against PBS and concentrated in 15 ml Biomax 10K NMWL Ultrafree 15 centrifugal filter device (Millipore Corporation, Bedford, Mass.). Protein concentrations were determined using Coomassie Plus Protein Assay Reagent (Pierce, Ill.). The purified APEX-1Ig and APEX-2mIg proteins were analyzed by SDS-PAGE. APEX-1Ig had slightly higher electrophoretic mobility than APEX-2mIg (FIG. 12).

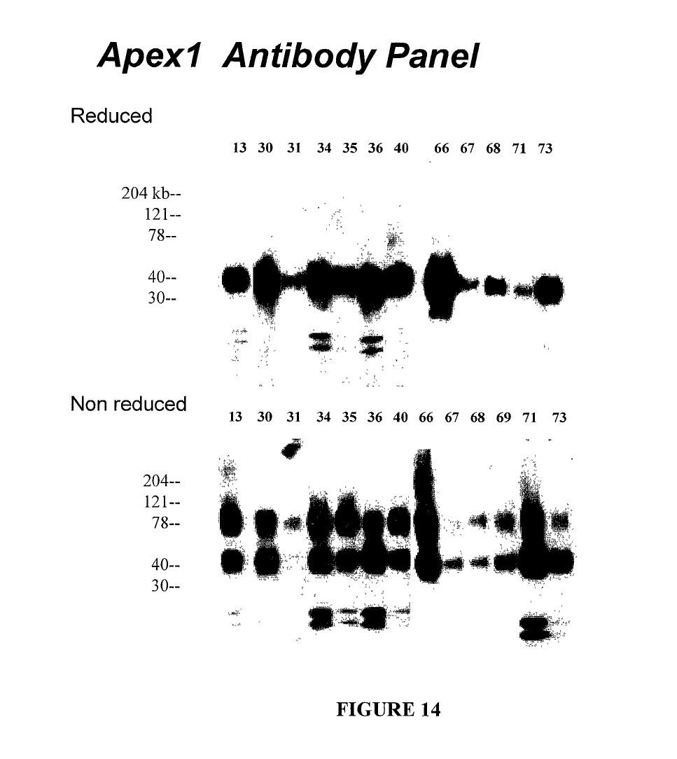

The human IgG1 construct included an amino-terminal thrombin cleavage site (Hollenbaugh et al., 1995), allowing separation of APEX-1 extracellular domain from IgG1. IgG1 was cleaved with thrombin (Sigma, St. Louis, Mo.) at 50:1 (w/w) Apex-1Ig:thrombin ratio for 1 hour at room temperature. Free IgG1 and non-cleaved Apex-1Ig were removed by adsorption to protein A and purified APEX-1 protein was further characterized by Western Blot analysis (described below, FIG. 14) using the monoclonal antibodies prepared by the invention (described in Example 5).

Expression of APEX-1Ig in Sf9 Cells

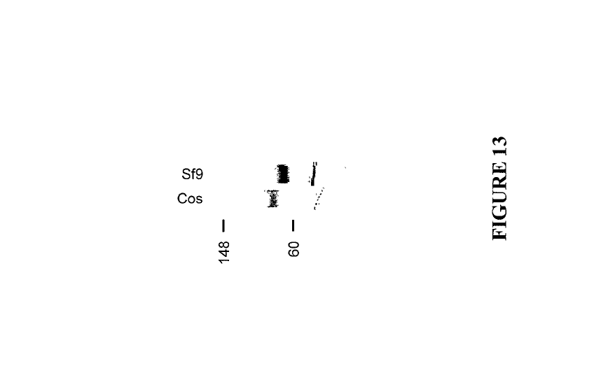

The cDNA encoding the APEX-1Ig (described above) was cloned into a modified pFastbac vector (Gibco-BRL Life Technologies, Gaithersburg, Md.) for baculovirus expression in Sf9 (Spodoptera frugiperda) insect cells. The entire Apex-1Ig cDNA was excised using HindIII and Xba1 restriction endonucleases, and ligated into the modified pFastbac vector cut with HindIII and Xba1. A Recombinant virus was obtained following transformation of DH10 Bac cells with pFastbac-Apex-1hIg vector. Sf9 cells (2×10⁶/ml) were infected with the recombinant virus (1/1000 dilution of PIII virus stock) and grown for 24 hr at 27 C. in EX-CELL 420 insect serum free medium (JRH Bioscience, Lenexa, Kans.). Cell free-supernatant was collected and passed over protein A-sepharose to purify the APEX-1Ig. APEX-1Ig produced by Sf9 cells had slightly increased electrophoretic mobility than that of APEX-1Ig produced by COS cells (FIG. 13), probably due to differences in protein glycosylation in the two expression systems.

Western Blot Analysis

APEX-1 extracellular domain (1 ug/lane) was pretreated in sample buffer with (reduced) or without (non-reduced) 2-mercaptoethanol, boiled for 5 min and loaded onto 4-20% Tris/Glycine gels (Novex, San Diego, Calif.) for SDS-PAGE. Following electrophoresis, proteins were transferred onto nitrocellulose membranes (BioRad, San Diego, Calif.). Membranes were blocked overnight in 5% non-fat milk powder/0.1% Tween-20/Tris buffered saline (T-TBS). Anti-APEX-1 mAb (hybridoma culture supernatant) were used to detect the extracellular domain of APEX-1. Bound antibody was visualized using HRP-conjugated donkey anti-mouse IgG (Jackson ImmunoResearch, Inc., West Grove, Pa.) and the Enhanced Chemiluminescence reagent (Amersham, Arlington Heights, Ill.) with Kodak X-Omat film.

Results

Fusion proteins including the extracellular domains of APEX-1 and APEX-2 fused to either human IgG1 (APEX-1Ig) or mouse IgG2a (APEX-2mIg) were produced as described above. Under reducing conditions, APEX-1Ig exhibited a molecular mass of 75-80 kDa, with APEX-2mIg migrating slightly faster corresponding to a molecular mass of 70-75 kDa (FIG. 12). Monoclonal antibodies against APEX-1 raised by immunizing mice with the APEX-1 recombinant extracellular domains, produced by cleavage of APEX-1Ig with thrombin, were used for Western Blot analysis of recombinanat APEX-1 Ig fusion protein so produced. The extracellular domain exhibiteda molecular weight of approximately 80 kDa under non-reducing conditions, and 40 kDa under reducing conditions, indicating the presence of disulfide bonds in the extracellular domain as produced by the cleaved fusion protein. The extracellular domain was recognized by a panel of monoclonal antibodies against APEX-1 (FIG. 14).

### EXAMPLE 5

Monoclonal Antibody Preparation

Female BALB/c mice were injected intraperitoneally at day 0 and day 21 with recombinant, COS produced, full-length extracellular domain of Apex-1 (50 μg) in RIBI adjuvant (Ribi Immunochem Research, Inc, Hamilton, Mont.). At day 31, serum (100 μl) was obtained to test for the presence of specific antibodies. At day 35, the mice received a final injection i.v. of 50 μg in PBS of Apex-1 protein. Three days after the last injection, mice were sacrificed and spleen cells were fused to the X63-Ag8-653 myeloma fusion partner at a ratio of 5:1 using 50% polyethylene glycol as previously described (Starling et. al. 1996). The fused cells were resuspended in complete IMDM medium supplemented with 2 mM glutamine, hypoxanthine (0.1 mM), aminopterin (0.01 mM), thymidine (0.016 mM) and 10% ORIGEN hybridoma cloning factor (IGEN International, Inc. Gaithersburg, Md.). The fused cells were then distributed between the wells of 96-well tissue culture plates, so that each well contained 1 growing hybrid on average.

After 10-14 days the supernatants of the hybridoma populations were screened for specific antibody production. Cells were grown and wells assayed for binding activity to Apex-1 but not an irrelevant human Ig fusion protein to remove reactivity against contaminating human Ig-specific hybridoma cells. Hybridoma cells secreting Apex-1Ig specific mAb were cloned (once) by limiting dilution (in IMDM containing 10% ORIGEN cloning factor and 2 mM glutamine) and expanded in tissue culture plates. Tissue culture supernatants were used as a source of unpurified mAb for western blotting experiments.

### EXAMPLE 6

Expression of Full-length APEX-1 and APEX-2 Polypeptides

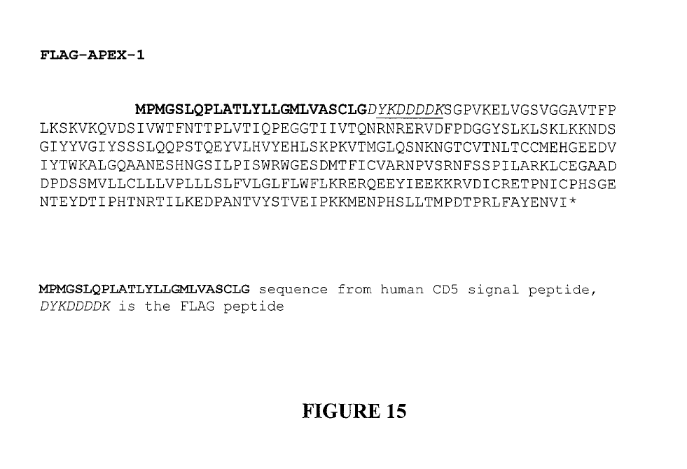

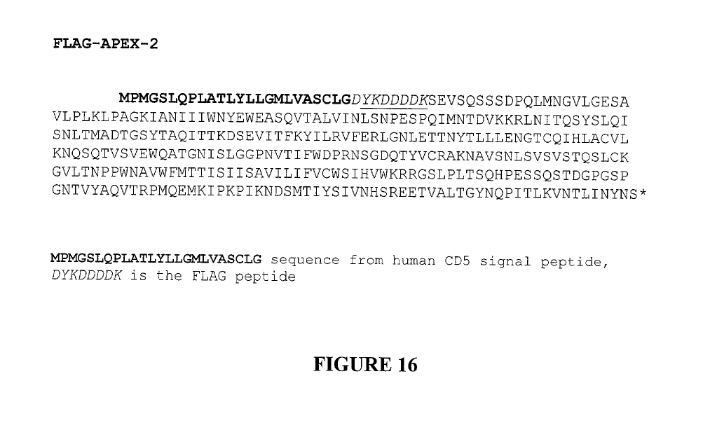

apex-1 and apex-2 cDNAs encoding for the full length mature polypeptides (excluding the sequences encoding native signal sequences) were cloned into pCDNA3 (Invitrogen, Carlsbad, Calif.) with cDNA encoding for the human CD5 signal peptide (Accession #X04391, amino acid sequence MPMGSLQPLATLYLLGMLVASCLG) and the FLAG tag peptide (DYKDDDDK) 5′ to the Apex sequence. FLAG sequences were added to the forward (5′) primers, to generate sequences including the following, CD5 Signal peptide-FLAG-APEX-1, and CD5 Signal peptide-FLAG-APEX-2-encoding sequences. The cDNAs were generated by PCR using primer sequences for apex-1: forward primer SpeFlagA1fp CGG ACT AGT GAC TAC AAG GAC GAC GAT GAC AAG TCT GGA CCC GTG AAA GAG CTG GTC GGT TCC (SEQ ID NO.:39), reverse primer A1Xbarp tgc tct aga cac tgc tgt cta gat aac att ctc ata ggc (SEQ ID NO.:40); apex-2 forward primer SpeFlagA2fp CGG ACT AGT GAC TAC AAG GAC GAC GAT GAC AAG AGT GAA GTT TCA CAG AGC AGC TCA GAC CCC (SEQ ID NO.:41), reverse primer A2xbarp tgc tct aga caa gtc act gca gtg ctc ttc ctt cag gag (SEQ ID NO.:42). PCR products were digested with the restriction endonucleases Spe1 and Xba1 and ligated to pCDNA3 that had been digested with Spe1 and Xba1. Ligated products were used to transform DH5a E. coli, which were grown on agar containing 100 ug/ml ampicillin. Cloned cDNAs were obtained from the bacteria by standard SDS lysis and purification on QIAGEN miniprep columns. The DNAs were sequenced to ensure fidelity of the PCR reaction. The encoded amino acid sequences of Flag-APEX-1 and Flag-APEX-2 are shown in FIGS. 15 and 16 respectively. The cDNAs were transfected into COS cells for transient expression or into human or mouse cell lines for stable expression using the antibiotic geneticin (Gibco-BRL Life Technologies) as a selection agent.

Cell surface expression can be detected by binding of specific antibody to either APEX-1 or APEX-2, or by M2 antibody (Sigma, St. Louis, Mo.) against the FLAG tag.

### EXAMPLE 7

Polyclonal Antibody Production

NZW rabbits were immunized with either the extracellular domains of Apex-1 or Apex-2mIg in Freund's Complete Adjuvant (initial immunization) or Freund's Incomplete Adjuvant (boosts). Following a series of immunizations, rabbits were bleed and sera were collected and processed by centrifugation. Sera will be adsorbed against human and mouse immunoglobulins to remove antibody recognizing the Ig portion of the fusion proteins used as immunogens. Antisera are to be used in functional studies and for cell surface staining.

Various publications are cited herein that are hereby incorporated by reference in their entirety.

As will be apparent to those skilled in the art in which the invention is addressed, the present invention may be embodied in forms other than those specifically disclosed without departing from the spirit or potential characteristics of the invention. Particular embodiments of the present invention described above are therefore to be considered in all respects as illustrative and not restrictive. The scope of the invention is as set forth in the appended claims and equivalents thereof, rather than being limited to the examples contained in the foregoing description.

References

1. Hahn, William et al., “CD2: A Multifunctional Coreceptor Involved in T-Cell Adhesion and Activation,” Lymphocyte Adhesion Molecules, 1993, Chap. 5:105-32.

2. Williams, Alan F. and A. Neil Barclay, “The Immunoglobulin Superfamily—Domains for Cell Surface Recognition,” Ann. Rev. Immunol., 1988, 6:381-405.

3. Dustin, Michael L. and Timothy A. Springer, “Role of Lymphocyte Adhesion Receptors in Transient Interactions and Cell Locomotion,” Ann. Rev. Immunol., 1991, 9:27-66.

4. Wingren, Anette Gjörloff et al., “T Cell Activation Pathways: B7, LFA-3, and ICAM-1 Shape Unique T Cell Profiles,” Critical Reviews in Immunology, 1995, 15(3&4):235-53.

5. Songyang, Zhou et al., “SH2 Domains Recognize Specific Phosphopeptide Sequences,” Cell, Mar. 12, 1993, 72:767-78.

6. Cocks, Benjamin G. et al., “A Novel Receptor Involved in T-Cell Activation,” Nature, Jul. 20, 1995, 376:260-3.

7. De la Fuente, Miguel Angel et al., “CD84 Leukocyte Antigen is a New Member of the Ig Superfamily,” Blood, Sep. 15, 1997, 90:2398-2405.

8. Songyang, Z. et al., “Specific Motifs Recognized by the SH2 Domains of Csk, 3BP2, fps/fes, GRB-2, HCP, SHC, Syk and Vav,” Molecular and Cellular Biology, April 1994, 14(4):2777-85.

What is claimed is:

1. An isolated nucleic acid molecule encoding APEX-1.

2. The isolated nucleic acid molecule of claim 1, wherein the molecule begins with a guanine (g) at position 1 and ends with an adenine (a) at position 2704 as shown in SEQ ID NO. 1.

3. The isolated nucleic acid molecule of claim 1, wherein APEX-1 has an amino acid sequence shown in SEQ ID NO. 4.

4. The isolated nucleic acid molecule of claim 3, wherein the amino acid sequence is encoded by a nucleotide sequence beginning with adenine (a) at position 42 and ending with guanine (g) at position 1049 as shown in SEQ ID NO. 1.

5. The isolated nucleic acid molecule of claim 1, wherein APEX-1 has an extracellular domain encoded by nucleotide sequences beginning with thymine (t) at position 108 and ending with cytosine (c) at position 716 as shown in SEQ ID NO. 1.

6. An isolated nucleic acid molecule encoding APEX-2.

7. The isolated nucleic acid molecule of claim 6, wherein the molecule begins with thymine (t) at position 1 and ends with thymine (t) at position 1516 as shown in SEQ ID NO.2, or a fragment thereof.

8. The isolated nucleic acid molecule of claim 6, wherein APEX-2 has an amino acid sequence shown in SEQ ID NO. 5.

9. The isolated nucleic acid molecule of claim 8, wherein the amino acid sequence is encoded by a nucleotide sequence beginning with adenine (a) at position 162 and ending with adenine at position 1217 as shown in SEQ ID NO. 2.

10. The isolated nucleic acid molecule of claim 6, wherein APEX-2 has an extracellular domain encoded by nucleotide sequences beginning at adenine (a) at position 249 and ending with guanine (g) at position 875 as shown in SEQ ID NO. 2.

11. An isolated nucleic acid molecule encoding APEX-3.

12. The isolated nucleic acid molecule of claim 11, wherein the molecule begins with guanine (g) at position 1 and ends with guanine (g) at position 1408 as shown in SEQ ID. NO. 3.

13. The isolated nucleic acid molecule of claim 11, wherein APEX-3 has an amino acid sequence shown in SEQ ID NO. 6.

14. The isolated nucleic acid molecule of claim 13, wherein the amino acid sequence is encoded by a nucleotide sequence beginning with adenine (a) at position 115 and ending with adenine at position 972 as shown in SEQ ID NO. 3.

15. An isolated polynucleotide variant having at least 70% polynucleotide sequence identity to the polynucleotide of claim 1, 6, or 11.

16. An isolated polynucleotide which hybridizes under stringent conditions to the complement of polynucleotide of claim 1, 6, or 11.

17. An isolated nucleic acid molecule comprising a nucleotide sequence which is complementary to the isolated nucleic acid molecule of claim 1, 6, or 11.

18. The isolated nucleic acid molecule of claim 1, 6, or 11 which is DNA or RNA.

19. The isolated nucleic acid molecule of claim 18, wherein the DNA is cDNA.

20. The isolated nucleic acid of claim 18, wherein the RNA is mRNA.

21. The isolated nucleic acid molecule of claim 1, 6, or 11 which is labeled with a detectable marker.

22. The nucleic acid molecule of claim 21, wherein the detectable marker is selected from the group consisting of a radioisotope, a fluorescent compound, a bioluminescent compound, a chemiluminescent compound, a metal chelator and an enzyme.

23. A vector comprising the nucleotide sequence of claim 1, 6, or 11.

24. A host vector system comprising the vector of claim 23 in a suitable host cell.

25. The host vector system of claim 24, wherein the suitable host is a bacterial cell.

26. The host vector system of claim 24, wherein the suitable host is an eukaryotic cell.

27. An isolated protein designated APEX-1 or APEX-2, comprising an extracellular domain having at least one Ig-like structure and at least one N-glycosylation site, a transmembrane domain, and a cytoplasmic domain having at least one SH2-binding motif.

28. The isolated protein designated APEX-1 of claim 27, having the amino acid sequence beginning at Met at position 1 and ending at Ile at position 335 as shown in SEQ. ID. No. 4, or fragments thereof, the protein or the fragments having APEX activity.

29. The isolated protein designated APEX-2 of claim 27, having the amino acid sequence beginning with Met at position 1 and ending at Ser at position 351 as shown in SEQ. ID. NO. 5, or fragments thereof, the protein or the fragments having APEX activity.

30. An isolated protein designated APEX-3 consisting of the amino acid sequence beginning with Met at position 1 and ending at Pro at position 285 as shown in SEQ. ID. NO. 6.

31. An antibody which recognizes and binds to the isolated protein of claim 27 or 30, or a fragment thereof having APEX activity.

32. A Fab′, F(ab)2′, or Fv fragment of the antibody of claim 31.

33. The antibody of claim 31, which is a monoclonal antibody.

34. The antibody of claim 33, which is 40-A10-G3 (ATCC Accession No.).

35. The antibody of claim 33, which is 66-H2-E5 (ATCC Accession No.).

36. The antibody of claim 33, which is 68-F12-G6 (ATCC Accession No.).

37. The antibody of claim 33, which is 71-E9-F10 (ATCC Accession No.).

38. The antibody of claim 31, which is a polyclonal antibody.

39. The antibody of claim 31, wherein the antibody is a chimeric antibody having a murine antigen-binding site and a humanized region that regulates effector functions.

40. The antibody of claim 31 which is labeled with a detectable marker.

41. The antibody of claim 40, wherein the detectable marker is selected from the group consisting of a radioisotope, a fluorescent compound, a bioluminescent compound, a chemiluminescent compound, a metal chelator and an enzyme.

42. A method of producing an APEX protein comprising:
a) culturing the host-vector system of claim 24 under suitable conditions so as to produce the APEX protein; and
b) recovering the APEX protein so produced.

43. An APEX protein produced by the method of claim 42.

44. A soluble APEX protein having a first amino acid sequence corresponding to an extracellular domain of an APEX protein and a second amino acid sequence corresponding to a moiety that alters the solubility of said APEX protein.

45. The soluble APEX protein of claim 44, wherein said moiety is an immunoglobulin constant region.

46. The soluble APEX protein of claim 44, wherein the APEX protein is APEX-1, APEX-2, or APEX-3.

47. A method for identifying a molecule in a sample which specifically binds an APEX protein, the method comprising:
(a) contacting the APEX protein with the sample under suitable conditions so as to obtain a complex having the APEX protein and the molecule;
(b) recovering the complex; and
(c) separating the APEX protein from the molecule in the complex and identifying the molecule so separated.

48. A method of of claim 47, wherein the sample is a tissue, e.g., brain, bone marrow, heart, kidney, liver, lung, lymph node, pancreas, placenta, skeletal muscle, thymus.

49. A method of claim 47, wherein the sample is a biological fluid, e.g., blood, urine, plasma, serum.

50. A nucleic acid molecule having a nucleotide sequence selected from a group consisting of SEQ ID NO: 7 to SEQ ID NO: 42.

51. A pharmaceutical composition comprising the APEX protein of claim 28, 29, or 30, and an acceptable carrier.

52. A pharmaceutical composition comprising an antibody or a antibody fragment thereof, that recognizes an APEX protein, and an acceptable carrier.
